(19) United States
(12) Patent Application Publication
(10) Pub. No.: US 2002/0168685 A1
(43) Pub. Date: Nov. 14, 2002

(54) Covalent chemical inducers of protein dimerization and their uses in high throughput binding screens

(76) Inventor: Virginia W. Cornish, New York, NY (US)

Correspondence Address: John P. White, Cooper & Dunham LLP, 1185 Avenue of the Americas, New York, NY, 10036, US

(21) Appl. No.: 10/056,874

(22) Filed: Jan. 24, 2002

Related U.S. Application Data
(63) Continuation-in-part of application No. 09/768,474, filed on Jan. 24, 2001.

(51) Int. Cl. G01N 33/53; C07H 19/22; C07H 19/073; C07H 19/067; C 07D 5 1/14; C07D 475/08

(52) U.S. Cl. 435/7.1; 536/27.13; 536/28.53; 540/222; 544/259

(57) ABSTRACT

Described are compounds having the formula:

H1-Y-H2

where H1 is a substrate capable of selectively binding to a first receptor; where H2 is a substrate capable of selectively binding to and selectively forming a covalent bond with a second receptor; and wherein Y is a moiety providing a covalent linkage between H1 and H2, which may be present or absent, and when absent, H1 is covalently linked to H2. Also described are uses of the compounds for in vivo screening of compounds and proteins.

This application is a continuation-in-part of U.S. Ser. No. 09/768,474, filed Jan. 24, 2001, the contents of which are hereby incorporated by reference.

This invention has been made with government support under National Science Foundation grants CHE-9626981, CHE-9977402, and CHE-9984928. Accordingly, the U.S. Government has certain rights in the invention.

Throughout this application, various publications are referenced by author or author and date. Full citations for these publications may be found listed alphabetically at the end of the specification immediately preceding the claims. The disclosures of these publications in their entireties are hereby incorporated by reference into this application in order to more fully describe the state of the art as known to those skilled therein as of the date of the invention described and claimed herein.

### FIELD OF INVENTION

This invention relates to high throughput screening of cDNA libraries.

### BACKGROUND OF THE INVENTION

The majority of known proteins were identified using traditional genetics or biochemistry. The availability of complete genome sequences for several organisms and the anticipation of the completion of the human genome project effectively make thousands of new proteins “known”. The problem, however, is that while thousands of new open reading frames (ORFs) have been identified, the functions of these proteins remain a mystery.

Sequence analysis is a powerful predictor of protein function, but many ORFs cannot be assigned by sequence analysis and experimental characterization is still required to ascertain protein function. There is tremendous interest in high-throughput approaches for testing cDNA libraries, which include thousands of unique ORFs, using genetic or biochemical screens. The hurdles are the same as in all high-throughput screening applications. First, cDNA libraries must be available in a format that is compatible with screening technologies and that allows rapid identification of individual cDNAs. Second, high-throughput screens must be developed.

Commercial cDNA libraries, tissue-specific cDNA libraries, and even cell-cycle-specific cDNA libraries from a variety of organisms are readily available. Over the past few years, these cDNA libraries have been adapted to several formats amenable to high-throughput screening.

Expression cloning relies on split-pool in vitro transcription/translation and has the advantage that it is compatible with many traditional biochemical assays. Winzler et al. used homologous recombination to engineer 2026 unique yeast strains—each containing a knock-out of a different ORF and replacing that ORF with a unique 20 base-pair tag. Several laboratories have reported specific yeast two-hybrid cDNA libraries, and many of these libraries are even distributed commercially (Clontech). Martzen et al. constructed 6144 individual yeast strains where each strain expresses a unique S. cerevisiae ORF-GST fusion protein under control of the P_{CUP1} promoter. Because of the facility of homologous recombination in S. cerevisiae, these cDNA libraries were prepared simply by co-transforming the cDNA library with the appropriate linearized vector. Thus, replicating these expression formats with different cDNA libraries is routine. The most common traditional genetic selection is lethality, or synthetic-lethality. A variety of phenotype-specific screens have also been employed. However, most of these are too time consuming for screening cDNA libraries. A few phenotype-specific selections have been reported. Screens and selections designed for the high-throughput screening of cDNA libraries have also been reported. One of the major applications envisioned for the yeast two-hybrid assay is the screening of cDNA libraries for protein-protein interactions. The success of the yeast three-hybrid assay suggests that it should also be possible to screen cDNA libraries for small-molecule-protein interactions. Another approach is to screen for changes in expression levels of individual cellular RNAs. In Genetic Footprinting, random Ty1 transposon insertions in genomic DNA are used as markers for changes in the expression levels of endogenous RNAs based on reverse transcription and gel electrophoresis. The use of unique oligonucleotide tags rather than Tyl transposon insertions facilitates rapid identification of individual RNAs. DNA microarrays, in which oligonucleotides corresponding to each individual ORF are synthesized on chips in a spatially-resolved format, have been used successfully in a number of recent applications. A recent report in which expression cloning identified a new family of uracil-DNA glycosylases from a Xenopus cDNA library based on in vitro binding assays suggests the importance of screening based on biochemical activity.

WO 01/53355 describes a number of screening approaches, including the use of small molecules to induce protein dimerization to screen cDNA libraries based on binding, or small molecules with cleavable linkers to screen cDNA libraries based on catalysis. The CID technology offers a promising approach to screening cDNA libraries based on function because a variety of activities can be assayed simply by changing one of the CID ligand/receptor pairs or by changing the bond between the CID ligands.

However, the existing CID approaches rely on 4 non-covalent interactions taking place simultaneously for the reporter protein to be activated. Specifically, 1) the DNA-binding protein-DNA interaction, 2) the 1^{st} ligand-receptor interaction, 3) the 2^{nd} ligand-receptor interaction, and 4) the activation domain-transcription machinery interaction. This approach is useful in certain types of screens.

An approach not employed by the reported CID screens is making a system with only 3 non-covalent interactions, yet still employing a small molecule as the CID.

### SUMMARY OF THE INVENTION

An embodiment of this invention are compounds having the formula:

H1-Y-H2

where H1 is a substrate capable of selectively binding to a first receptor; where H2 is a substrate capable of selectively binding to and selectively forming a covalent bond with a second receptor; and wherein Y is a moiety providing a covalent linkage between H1 and H2, which may be present or absent, and when absent, H1 is covalently linked to H2. Also described are cells for use with the compounds for in vivo screening of compounds and proteins.

In this compound, the 1^{st} ligand-receptor pair (Dex-GR in FIG. 13) is replaced with a small molecule-receptor pair that will form an irreversible covalent linkage, making a system with only 3 non-covalent interactions. Such an approach allows for the screening of small molecules to identify their cellular targets. This covalent CID system is used for screening the ligand receptor interaction, which used to be laborious work by using the photo cross linking, radio labeled ligand binding and affinity chromatography techniques. The covalent system is more sensitive than the Dex-Mtx system because the covalent bond gives zero k_{off} for the covalent ligand-protein binding pair and then the cut-off Kd of the whole system is enhanced.

### DESCRIPTION OF THE FIGURES

FIG. 1. The selection strategy. Proteins V and W do not interact (A) until a BOND links the handles H1 and H2 (B). The selection can be run in the forward direction to select for BOND formation or the reverse direction to select for BOND cleavage.

FIG. 2. The yeast three-hybrid system. The small molecule dexamethasone-FK506 (H1-H2) mediates the dimerization of the LexA-GR (glucocorticoid receptor) and B42-FKBP12 protein fusions. Dimerization of the DNA-binding protein LexA and the activation domain B42 activates transcription of the lacz reporter gene.

FIG. 3. The Model reaction. Cephalosporin hydrolysis by the 908R cephalosporinase.

FIG. 4. DEX-CEPHEM-FK506 retrosynthesis. Cephem 1 is commercially available. DEX-CO₂H is prepared via oxidation of the C₂₀ ∝-hydroxy ketone; FK506-CO₂H, via a cross-metathesis reaction with the C₂₁ allyl group.

FIG. 5. The chemical handles dexamethasone (A), FK506 (B), and methotrexate (C).

FIG. 6. The dexamethasone-methotrexate molecules synthesized. The diamine linkers are commercially available and vary in length and hydrophobicity.

FIG. 7. The Claisen rearrangement (A) and the Diels-Alder reaction (B) are both pericyclic reactions with six-membered transition states.

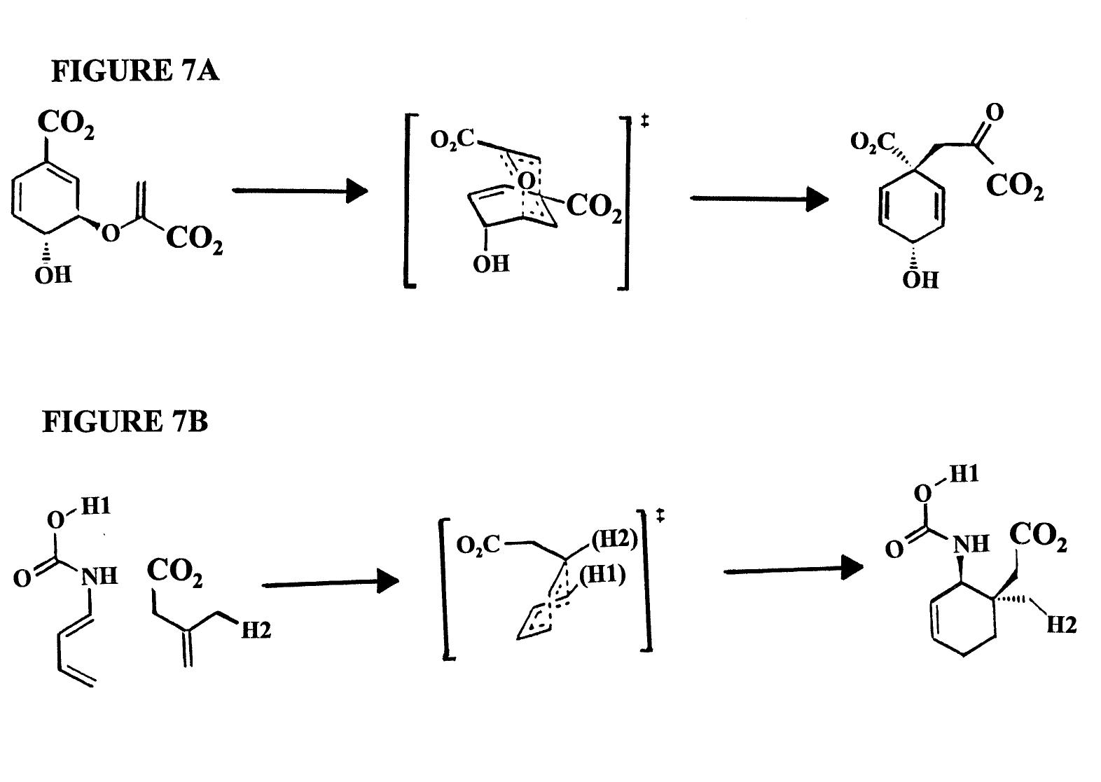

FIG. 8. The retro-synthesis of the diene (A) and the dienophile (B). A Curtius rearrangement is used to introduce the carbamyl linkage to H1 in the diene. (Overman) A Stille coupling is used to introduce the alkyl linkage to H2 in the dienophile. (Duchene) The cyclohexene product will be prepared through the cycloaddition of these two compounds.

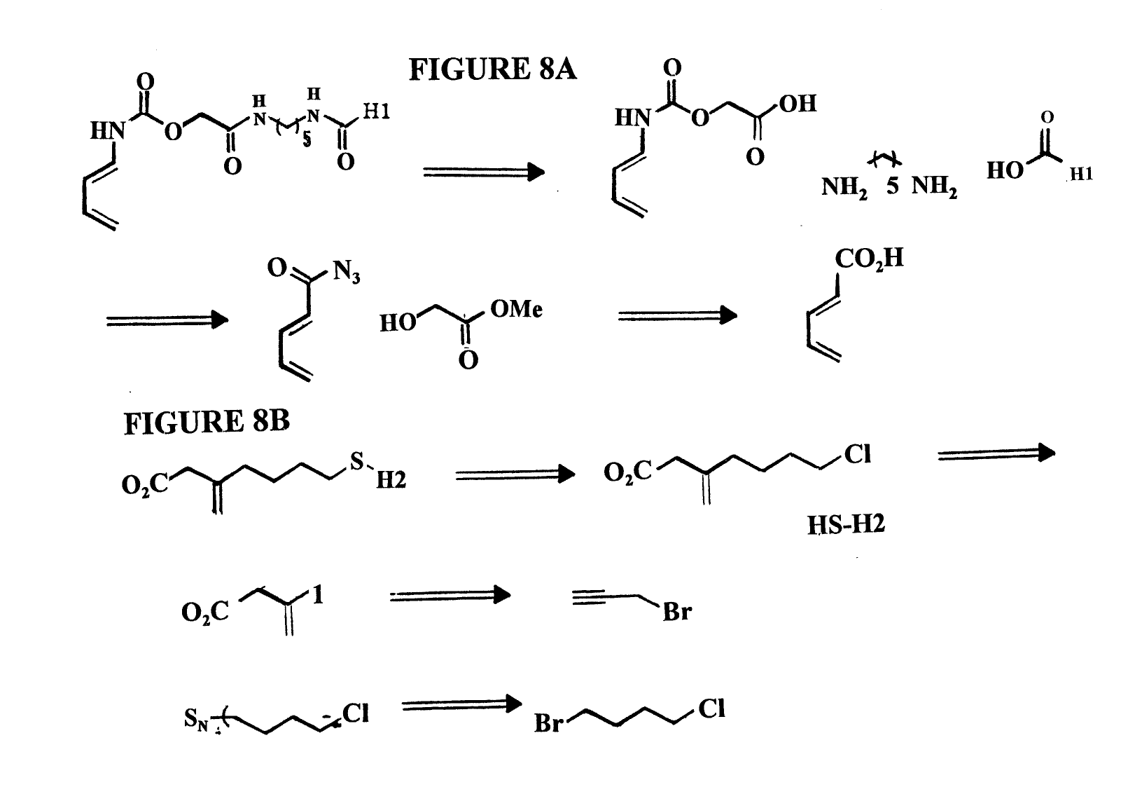

FIG. 9. Examples of DEX-DEX molecules synthesized to date.

FIG. 10. DEX-MTX retrosynthesis.

FIG. 11. Maps of the plasmids encoding the LexA-GR and B42-GR fusion proteins.

FIG. 12. Dex-cephem-Mtx retro-synthesis.

FIG. 13. Dex-Mtx protein dimerization system. A cell-permeable Dex-Mtx molecule is used to induce dimerization of LexA-GR and DHFR-B42 protein chimeras, activating transcription of a lacZ reporter gene.

FIG. 14. Cell based assays. Yeast cells containing LexA-GR and b42-DHFR fusion proteins and the lacZ reporter gene are grown on X-gal plates with or without Dex-Mtx. Dex-Mtx dimerizes the fusion proteins, activating lacZ transcription, hydrolyzing the chromogenic substrate X-gal, and turning the cells blue. Dex-Mtx is added directly to the media in the x-gal plate. The assay takes two to five days.

FIG. 15. X-gal plate assay of Dex-cephem-Mtx induced lacZ transcription. Yeast strains containing different LexA- and B42 chimeras, plus a lacZ reporter gene, were grown on X-gal indicator plates with or without Dex-cephem-MTX compounds: A, 1 μM Dex-MTX; B, 10 μM Dex-cephem-MTX; C, no small molecule. The strains that are dark (blue in original) even in the absence of small molecule (plate C) are positive controls on protein-protein interaction. The dark strains on plates A and B express LexA DHFR and B42-GR fusion proteins, and the white strains are negative controls, expressing only LexA and B42.

FIG. 16A. Plate BTC4 grown on 4 different plates after 72 hours. One plate has no small molecule, so just the positive controls should be dark. The other three plates all have either 10 uM DM1, 10 uM D8M, or 10 uM D10M.

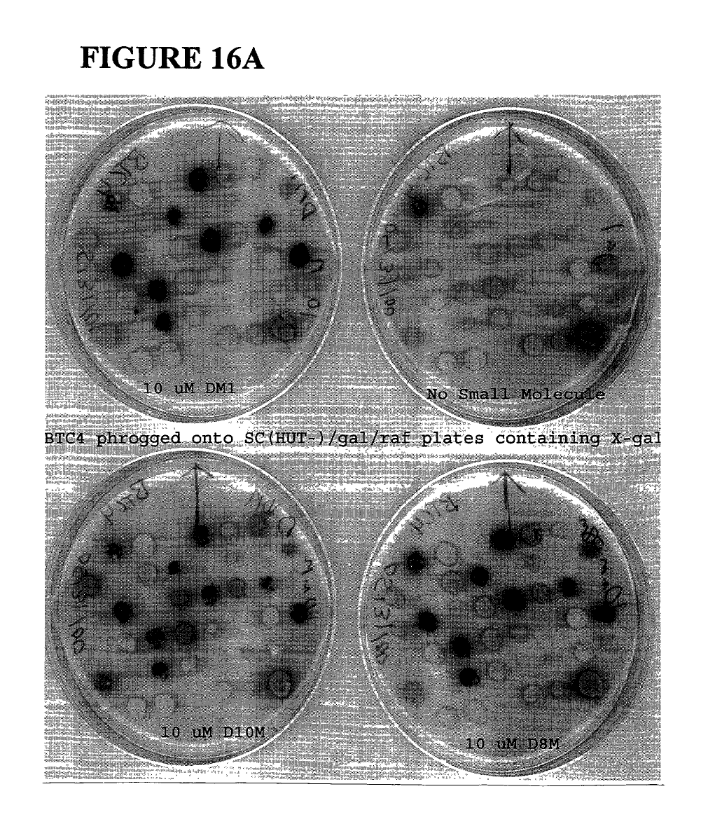

FIG. 16B is the plate map for plate BTC4.

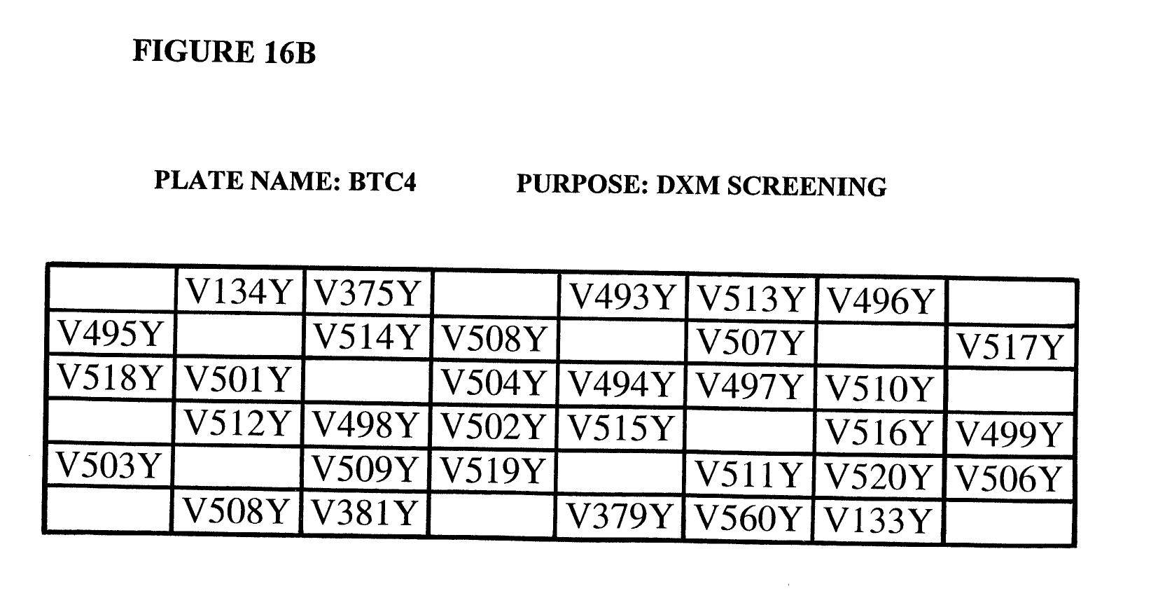

FIG. 17A. Plate BTC6 grown on 4 plates after 56 hours. Two top plates contain no small molecule, and the bottom two plates contain 10 uM D10M.

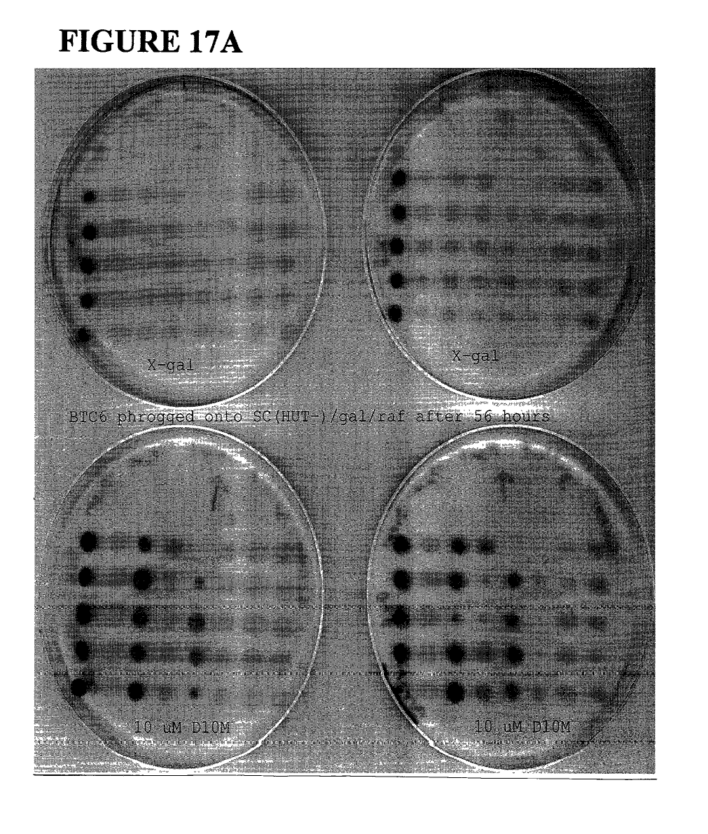

FIG. 17B shows plate BTC6 grown on 2 plates after 60 hours. Both plates contain 1 uM D8M.

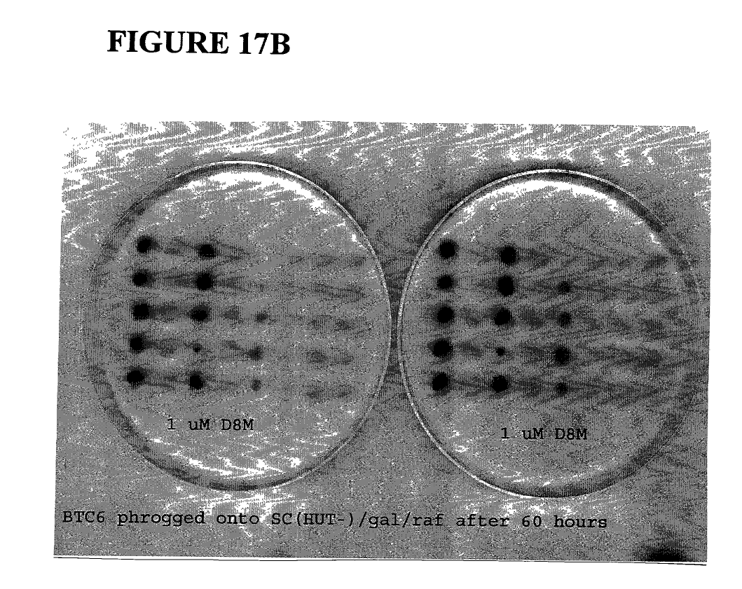

FIG. 17C shows the plate map for plate BTC6.

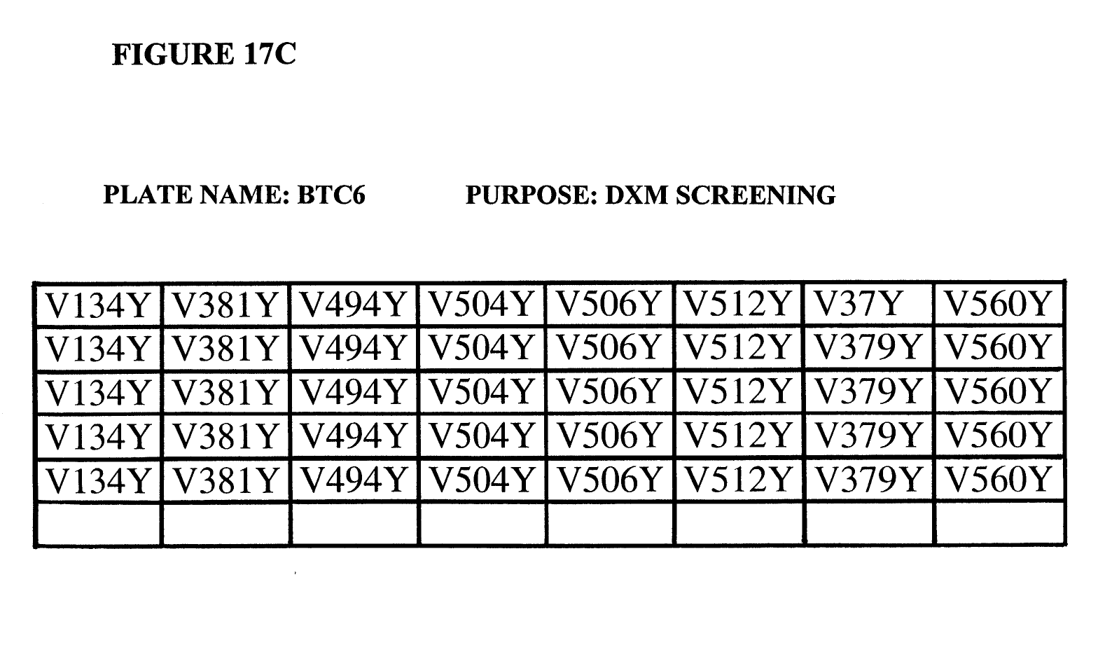

FIG. 18. The β-galactosidase activity of strain V494Y using varying concentrations of D8M.

FIG. 19. A screen for glycosidase activity. Dex-Mtx CIDs with cleavable oligosaccharide linkers used to assay the >3000 proteins in S. cerevisiae of unknown function for glycosidase activity. A yeast cDNA library is introduced into the selection strain. Only cells expressing active glycosidases cleave the oligosaccharide linker, disrupt ura3 transcription, and survive in the presence of 5-FOA.

FIG. 20. Proposed solid-phase synthesis of the Dex-Mtx glycosidase substrates. While the synthesis of Dex-(GlcNAc)₄-Mtx is shown, the synthesis is designed to allow the introduction of a variety of sugar monomers with both regio- and stereo-control.

FIG. 21. The synthesis route of Mtx-Cephem.

### DETAILED DESCRIPTION OF THE INVENTION

An embodiment of the invention is a compound having the formula:

H1-Y-H2

wherein H1 is a substrate capable of selectively binding to a first receptor;

wherein H2 is a substrate capable of selectively binding to and selectively forming a covalent bond with a second receptor; and

wherein Y is a moiety providing a covalent linkage between H1 and H2, which may be present or absent, and when absent, H1 is covalently linked to H2.

H1 may be a Methotrexate moiety or an analog thereof.

H2 may be a cephem moiety capable of selectively binding to and selectively forming a covalent bond with the penicillin-binding-protein (“PBP”). H2 may alternatively be a fluorouracil moiety capable of selectively binding to and selectively forming a covalent bond with the thymidine synthase (“TS”) enzyme.

The compound may have the structure:

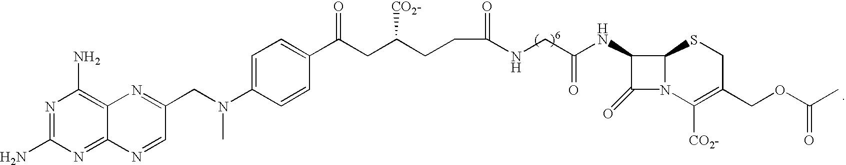

Another embodiment of the invention is a complex between the compound and a fusion protein, the fusion protein comprising a receptor domain which binds to the compound.

The fusion protein may further comprise a DNA-binding domain fused to the receptor domain, or a transcription activation domain fused to the receptor domain.

In the complex, the receptor domain may be dihydrofolate reductase (“DHFR”), penicillin-binding-protein (“PBP”), or thymidine synthase (“TS”) enzyme. The PBP may be the Streptomyces R61 PBP. The DHFR may be the E.coli DHFR (“eDHFR”).

The fusion protein in the complex may be eDHFR-LexA, R61-LexA, eDHFR-B42 or R61-B42.

Also described is cell comprising the complex.

The cell may comprise a DNA sequence which on transcription gives rise to a first fusion protein exogenous to the cell and a second fusion protein exogenous to the cell,

wherein the first fusion protein is a receptor domain fused with a DNA-binding domain; and

wherein the second fusion protein is a transcription activation domain fused to either a penicillin-binding-protein (“PBP”) or to a thymidine synthase (“TS”) enzyme.

In the cell, the receptor domain of the first fusion protein may be DHFR; the DNA-binding domain of the first fusion protein may be LexA; the transcription activation domain of the second fusion protein may be B42. In the cell, the PBP may be the Streptomyces R61 PBP.

The first fusion protein in the cell may be eDHFR-LexA, and the second fusion protein may be R61-B42.

The cell may be a yeast cell, a bacteria cell or a mammalian cell. In an embodiment, the cell is S. cerevisiae or E. coli.

Also disclosed is a method of dimerizing two fusion proteins inside a cell using the compound, the method comprising the steps of

a) providing a cell that expresses a first fusion protein which comprises a receptor domain that binds to H1, and a second fusion protein which comprises a receptor domain that binds to and forms a covalent bond with H2, and

b) contacting the compound with the cell so as to dimerize the two fusion proteins.

In the method, the receptor domain of the first fusion protein may be DHFR; the DNA-binding domain of the first fusion protein may be LexA; the transcription activation domain of the second fusion protein may be B42; the receptor domain of the second fusion protein may be a penicillin-binding-protein (“PBP”) or to a thymidine synthase (“TS”) enzyme. The PBP may be the Streptomyces R61 PBP.

In an embodiment of the method, the first fusion protein is eDHFR-LexA, and the second fusion protein is R61-B42.

Also disclosed is a method for identifying a molecule that binds a known target in a cell from a pool of candidate molecules, comprising:

(a) forming a screening molecule by covalently bonding each molecule in the pool of candidate molecules to a substrate capable of selectively binding to and selectively forming a covalent bond with a receptor;

(b) introducing the screening molecule into a cell culture comprising cells that express

a first fusion protein of a DNA-binding domain fused to a known target receptor domain against which the candidate molecule is screened,

a second fusion protein which comprises a receptor domain capable of binding to and forming a covalent bond with the screening molecule, and

a reporter gene wherein expression of the reporter gene is conditioned on the proximity of the first fusion protein to the second fusion protein;

(c) permitting the screening molecule to bind to the first fusion protein and to the second fusion protein, bringing te two fusion proteins in to proximity so as to activate the expression of the reporter gene;

(d) selecting the cell that expresses the reporter gene; and

(e) identifying the small molecule that binds the known target receptor.

The cell may be selected from the group consisting of insect cells, yeast cells, mammalian cell, and their lysates.

The parts of the fusion proteins are as described previously.

In the method, the molecule may be obtained from a combinatorial library.

The method may have steps (b)-(e) iteratively repeated in the presence of a preparation of random small molecules for competitive binding with the screening molecule so as to identify a molecule capable of competitively binding the known target receptor.

Also disclosed is a method for identifying an unknown target receptor to which a molecule is capable of binding in a cell, comprising:

(a) providing a screening molecule having a ligand which has a specificity for the unknown target receptor covalently bonded to a substrate capable of selectively binding to and selectively forming a covalent bond with a receptor;

(b) introducing the screening molecule into a cell which expresses

a first fusion protein of a DNA-binding domain fused to the unknown target receptor domain against which the candidate molecule is screened,

a second fusion protein which comprises a receptor domain capable of binding to and forming a covalent bond with the screening molecule, and

a reporter gene wherein expression of the reporter gene is conditioned on the proximity of the first fusion protein to the second fusion protein;

(c) permitting the screening molecule to bind to the first fusion protein and to the second fusion protein so as to activate the expression of the reporter gene;

(d) selecting which cell expresses the unknown target receptor; and

(e) identifying the unknown target receptor.

In the method, the unknown protein target is encoded by a DNA from the group consisting of genomicDNA, cDNA and syntheticDNA.

In the method, the ligand may have a known biological function.

Also disclosed are compounds having the formula:

H1-Y-H2

wherein H1 is Mtx or an analog thereof;

wherein H2 is a substrate capable of binding to a receptor, and

wherein Y is a moiety providing a covalent linkage between H1 and H2, which may be present or absent, and when absent, H1 is covalently linked to H2.

The specific structures of the compounds are as show below:

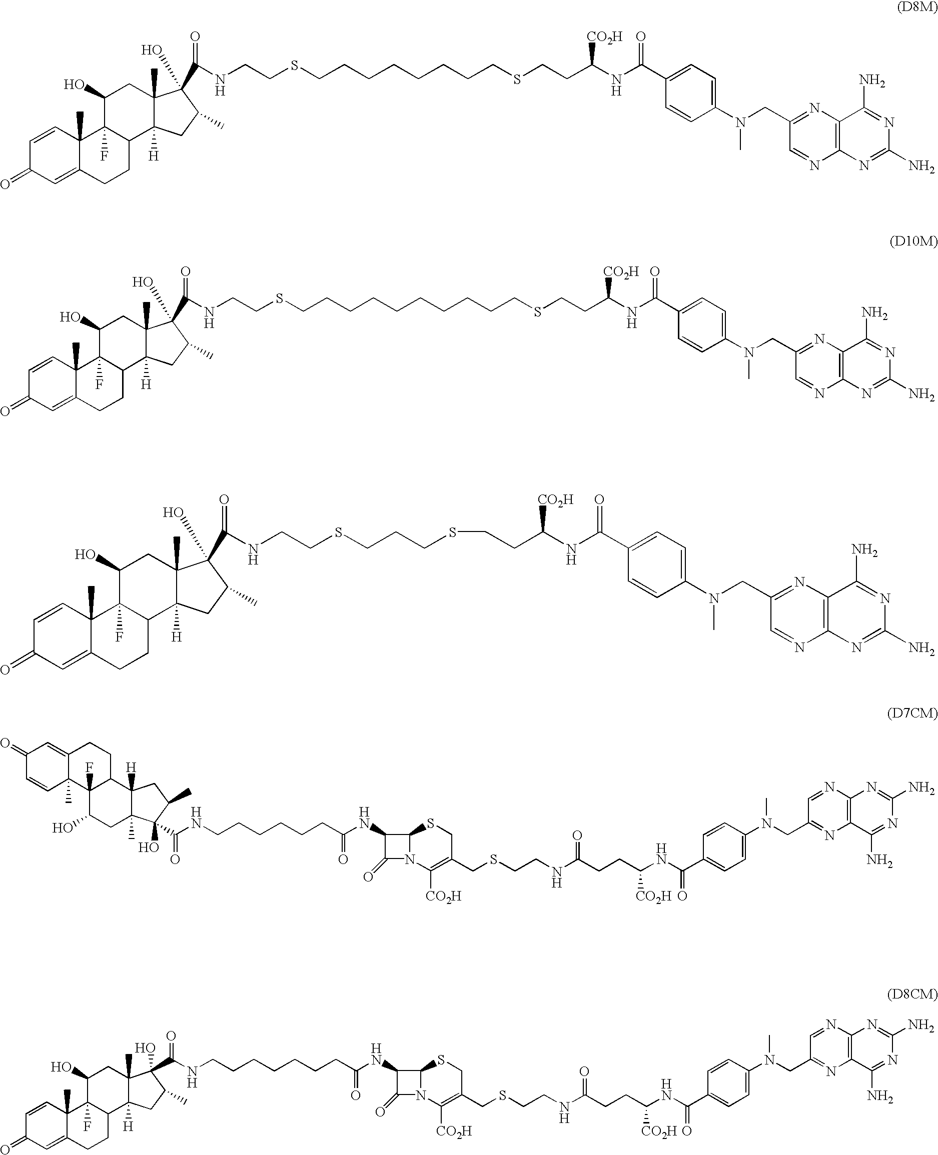

Also disclosed is a complex between the above compounds and a fusion protein which comprises a binding domain capable of binding to methotrexate, wherein H1 of the compound binds to the binding domain of the fusion protein.

In this complex, the binding domain is that of the DHFR receptor, or the fusion protein is DHFR-LexA or DHFR-B42.

Also disclosed is a cell comprising this complex, and a method for screening a cDNA library by identifying the expressed protein target, comprising:

(a) providing a screening molecule comprising a methotrexate moiety or an analog of methotrexate covalently bonded to a ligand which has a known specificity;

(b) introducing the screening molecule into a cell which expresses a first fusion protein comprising a binding domain capable of binding methotrexate, a second fusion protein comprising the expressed unknown protein target, and a reporter gene wherein expression of the reporter gene is conditioned on the proximity of the first fusion protein to the second fusion protein;

(c) permitting the screening molecule to bind to the first fusion protein and to the second fusion protein so as to activate the expression of the reporter gene;

(d) selecting which cell expresses the reporter gene; and

(e) identifying the unknown protein target and the corresponding cDNA.

In this method, the unknown protein target may be encoded by a DNA from the group consisting of genomicDNA, cDNA and syntheticDNA. Other elements are as described previously.

Also disclosed is a new protein cloned by the method.

In any embodiment of the invention, the cell may be selected from the group consisting of insect cells, yeast cells, mammalian cell, and their lysates. The first or the second fusion protein may comprise a transcription module selected from the group consisting of a DNA binding protein and a transcriptional activator. Also, the molecule may be obtained from a combinatorial library.

Any of the described methods can be adapted to determine the cellular function of a natural protein. The methods can also be adapted to identify the cellular targets of a drug, this method further comprising screening with the drug in question being part of the CID.

The described methods may also be adapted to identify new protein targets for pharmaceuticals.

The described methods may also be adapted for determining the function of a protein, this method further including screening with a natural cofactor being part of the CID.

The described methods may also be adapted for determining the function of a protein, this method further including screening with a natural substrate being part of the CID.

The described methods may also be adapted for screening a compound for the ability to inhibit a ligand-receptor interaction.

In any of the described embodiments, each of H1 and H2 is capable of binding to a receptor with a IC₅₀ of less than 100 nM. In a preferred embodiment, each of H1 and H2 is capable of binding to a receptor with a IC₅₀ of less than 10 nM. In the most preferred embodiment, each of H1 and H2 is capable of binding to a receptor with a IC₅₀ of less than 1 nM.

Each of H1 or H2 may be derived from a compound selected from the group consisting of steroids, hormones, nuclear receptor ligands, cofactors, antibiotics, sugars, enzyme inhibitors, and drugs.

Each of H1 and H2 may also represent a compound selected from the group consisting of dexamethasone, 3,5,3′-triiodothyronine, trans-retinoic acid, biotin, coumermycin, tetracycline, lactose, methotrexate, FK506, and FK506 analogs.

In any of the described methods, the cellular readout may be gene transcription, such that an increase in gene transcription indicates catalysis of bond formation by the protein to be screened.

In the described methods, the screening is performed by Fluorescence Associated Cell Sorting (FACS), or gene transcription markers selected from the group consisting of Green Fluorescence Protein, LacZ-β-galagctosidases, luciferase, antibiotic resistant β-lactamases, and yeast markers.

As discussed, the foregoing may be adapted to the determination of the binding specificity of biomolecules is important not only for understanding the mechanisms and pathways of biological systems, but also because this binding specificity provides information for the future development of therapeutic and diagnostic agents. This invention describes a cell-based assay for detecting binding activities of steroids to better understand their in vivo molecular recognition. Steroid hormones are essential for the regulation of salts and water in the body, for metabolism, and for the maturation and sexual development of males and females. Moreover, the development of several kinds of cancer has been linked directly to steroids as causative agents. Due to the necessary role that steroids play in bodily functions, it is important to learn about their interactions with cellular targets to understand how they demonstrate this dual behavior. The screen builds from existing technology for dimerizing proteins within cells, using chemical inducers of dimerization (“CID” or “CIDs”). By using a steroid as one of the ligands of the dimeric small-molecule CIDs, binding can be detected.

The foregoing embodiments of the subject invention may be accomplished according to the guidance which follows. Certain of the foregoing embodiments are exemplified. Sufficient guidance is provided for a skilled artisan to arrive at all of the embodiments of the subject invention.

Selection Strategy

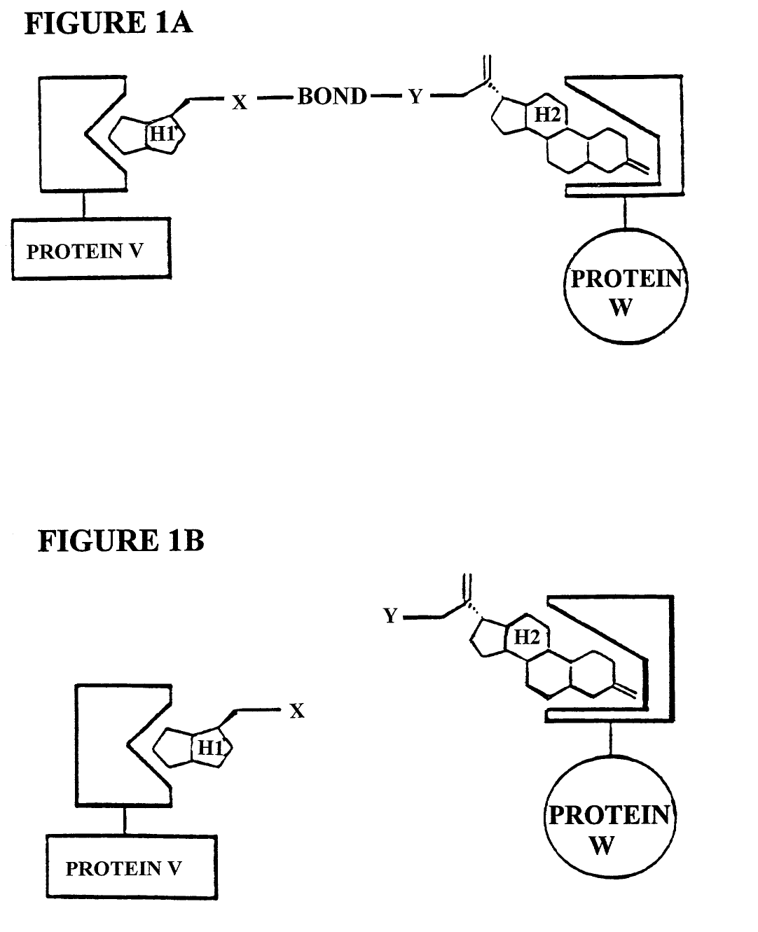

The selection strategy is based on existing methods for controlling protein dimerization in vivo using small molecules (FIG. 1). Several “chemical inducers of dimerization” have been reported showing that protein dimerization can be bridged by small molecules. (Spencer; Crabtree) Moreover, a number of techniques exist for translating the dimerization of two proteins to an in vivo screen or selection. (Hu 1990; Hu 1995; Fields; Gyuris; Johnsson; Rossi; Karimova) Taken together, this work establishes that it is feasible to use a small molecule H1-H2 to dimerize two fusion protein, reporter V-H1 receptor and reporter W-H2 receptor, generating a cellular read-out.

Disclosed a general method for screening a CDNA library based on the ability of members of that library to express a protein capable of binding to H1 or H2 or an ability of that protein to catalyze a reaction to either form or cleave the covalent coupling between H1 and H2. That is, the small-molecule H1-X-BOND-Y-H2 represented in FIG. 1 is used to mediate protein dimerization and hence a cellular signal. Then the polypeptide enzyme that binds to either H1 or H2 is selected. The selection is tied to the cellular “read-out” because only cells containing the polypeptide which binds will have the desired phenotype.

The strategy is both general and a direct selection for polypeptides which bind. The selection can be applied to a broad range of polypeptides because protein dimerization depends only on the H1 and H2 selected. It is a direct selection for the polypeptides because binding of H1 and H2 is necessary for protein dimerization. Also, this strategy does not limit the starting protein scaffold.

Preparation and Design of Handles “H1” and “H2”

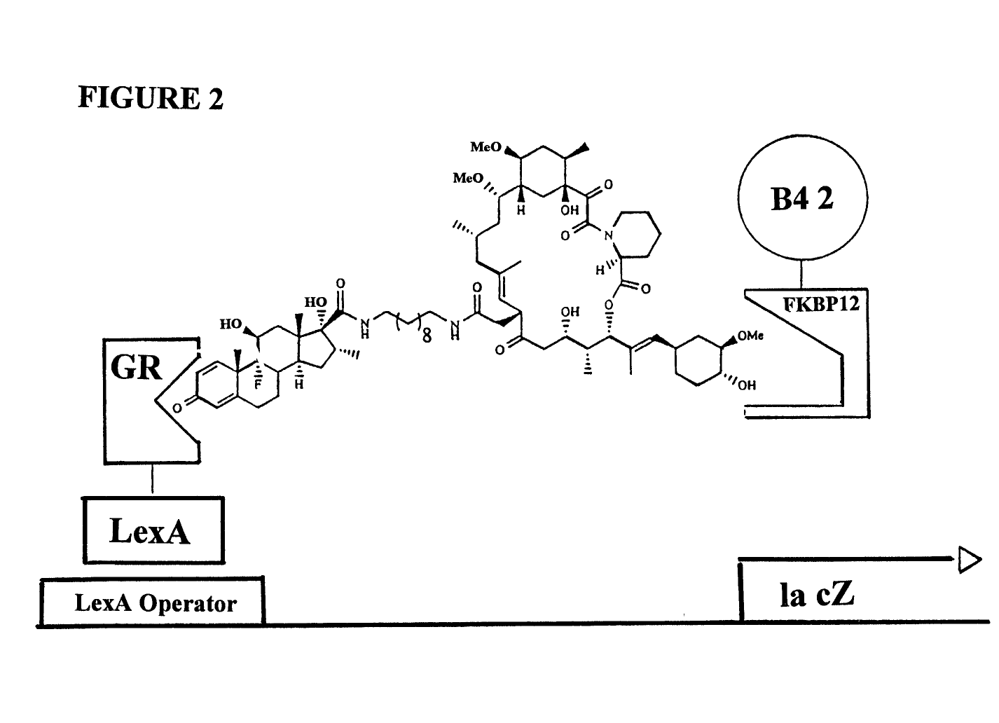

Ideally, a chemical handle should bind its receptor with high affinity (≦100 nM), cross cell membranes yet be inert to modification or degradation, be available in reasonable quantities, and present a convenient side-chain for routine chemical derivatization that does not disrupt receptor binding. Again, we build from DEX-FK506 (H1-H2) mediated dimerization of LexA-rGR and B42-FKBP12 (FIG. 2) (Licitra).

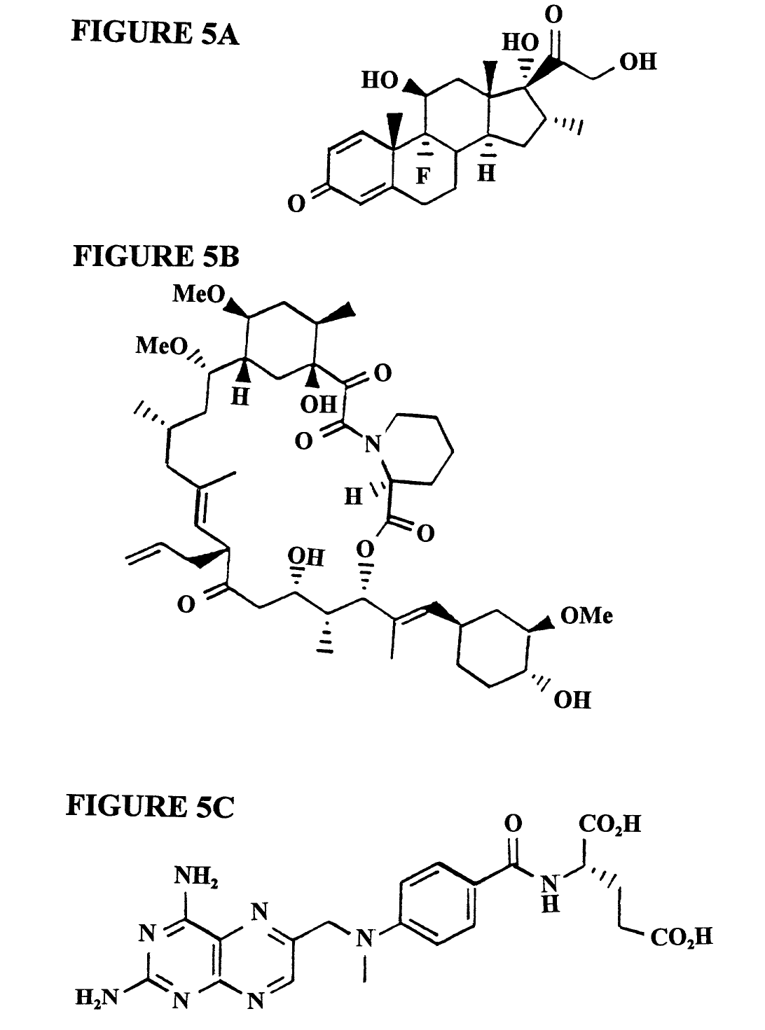

Dexamethasone (DEX) is a very attractive chemical handle H1 (FIG. 5A). DEX binds rat glucocorticoid receptor (GR) with a KD of 5 nM, (Chakraborti) can regulate the in vivo activity and nuclear localization of GR fusion proteins (Picard), and is commercially available. Affinity columns for rGR have been prepared via the C₂₀ ∝-hydroxy ketone of dexamethasone. (Govindan; Manz).

The antibacterial and anticancer drug methotrexate (MTX) is used in place of FK506 as the chemical handle H2 (FIGS. 5B, 5C) FK506 is not available in large quantities, coupling via the C₂₁ allyl group requires several chemical transformations including silyl protection of FK506, (Spencer; Pruschy) and FK506 is both acid and base-sensitive. (Wagner; Coleman) MTX, on the other hand, is commercially available and can be modified selectively at its γ-carboxylate without disrupting dihydrofolate reductase (DHFR) binding. (Kralovec; Bolin) Even though MTX inhibits DHFR with pM affinity, (Bolin; Sasso) both E. coli and S. cerevisiae grow in the presence of MTX when supplemented with appropriate nutrients. (Huang)

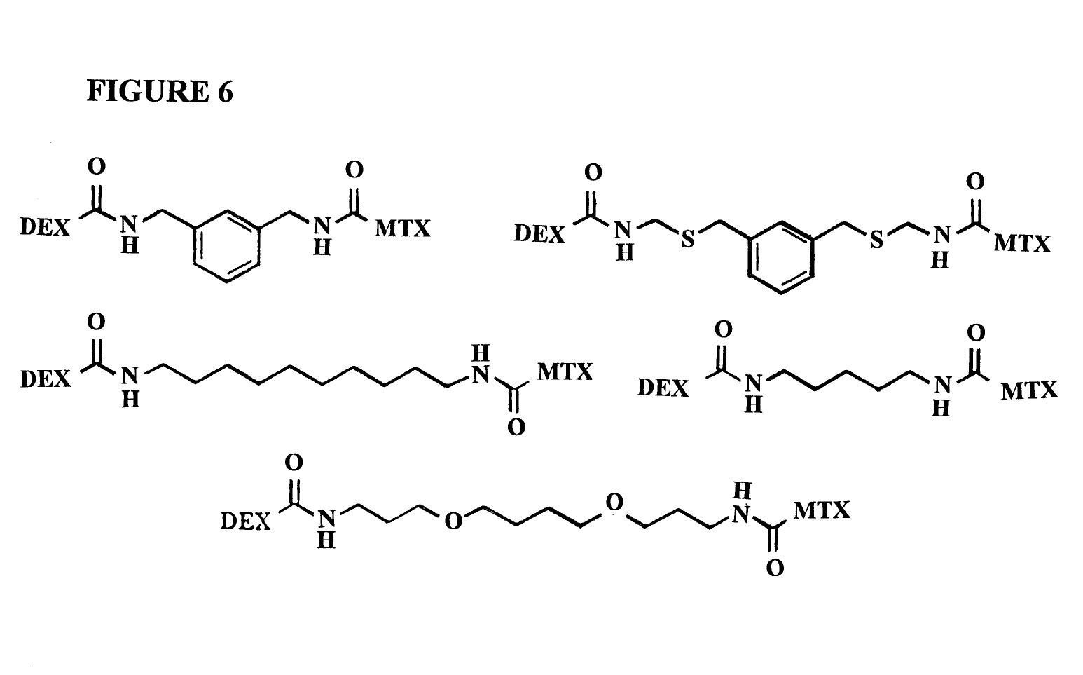

The ability of DEX-MTX to mediate the dimerization of LexA-rGR and B42-DHFR is tested by (1) synthesis of a series of DEX-MTX molecules with simple diamine linkers (FIG. 6); and (2) showing that DEX-MTX can dimerize LexA-rGR and B42-DHFR based on lacZ transcription and that both DEX and MTX uncoupled, can, competitively disrupt this dimerization. Cell permeable chemical handles that can be prepared readily and that are efficient at inducing protein dimerization not only are essential to the robustness of this selection methodology but also should find broad use as chemical inducers of protein dimerization.

Dexamethasone (DEX) and the glucocorticoid receptor (GR) present a particularly attractive chemical handle/receptor pair. Dexamethasone is the cortical steroid with the highest affinity for the rat Glucocorticoid Receptor. The rGR binds DEX with a K_{D} of 5 nM, and mutants of rGR have been isolated with up to 10-fold higher affinity for DEX. (Chakraborti) The steroid dexamethasone has been used extensively as a cell-permeable small molecule to regulate the in vivo activity and nuclear localization of GR fusion proteins. (Picard) This work firmly establishes that DEX is cell permeable and is not modified or broken down in the cell. Recently, there has been one report of a yeast “three-hybrid” system in which a GR-DNA-binding protein fusion and a FKBP12-transcription activation domain fusion could be dimerized by the small molecule DEX-FK506 (FIG. 2). Dexamethasone is commercially available in large quantities. Affinity columns for rGR have been prepared via oxidation of the C₂₀ a-hydroxy ketone of DEX to the corresponding carboxylic acid. (Govindan, Manz)

Methotrexate (MTX) inhibition of dihydrofolate reductase (DHFR) is one of the textbook examples of high-affinity ligand binding. The interaction between MTX and DHFR is extremely well characterized both biochemically and structurally. DHFR is a monomeric protein and binds MTX with picomolar affinity. (Bolin, Sasso) Even though MTX inhibits DHFR with such high affinity, both E. coli and S. cerevisiae grow in the presence of MTX when supplemented with appropriate nutrients. (Huang) The ability of MTX to serve both as an antibacterial and an anticancer agent is clear evidence that MTX has excellent pharmacokinetic properties. MTX is known to be imported into cells via a specific folate transporter protein. MTX is commercially available and can be synthesized readily from simple precursors. MTX can be modified selectively at its g-carboxylate without disrupting its interaction with DHFR. (Kralovec, Bolin) There are several examples reported where MTX has been modified via its g-carboxylate to prepare affinity columns and antibody conjugates.

Given the number of cellular pathways that depend on cascades of dynamic protein-protein interactions, the ability to regulate protein oligomerization in vivo with small molecules should have broad applications in medicine and basic science. The key to realizing the potential of these small molecules both for the catalysis screen in the laboratory and for these biomedical applications is developing H1-H2 molecules that can be prepared readily and are efficient at inducing protein dimerization in vivo.

Other handles H1 and H2 may be for example, steroids, such as the Dexamethasone used herein; enzyme inhibitors, such as Methotrexate used herein; drugs, such as KF5O6; hormones, such as the thyroid hormone 3,5,3′-triiodothyronine (structure below)

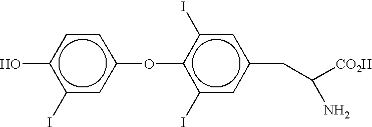

Ligands for nuclear receptors, such as retinoic acids, for example the structure below

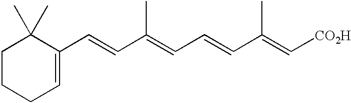

General cofactors, such as Biotin (structure below)

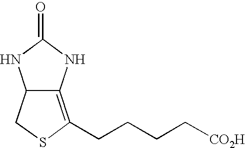

and antibiotics, such as Coumermycin (which can be used to induce protein dimerization according to Perlmutter et al., Nature 383, 178 (1996)).

Derivative of the mentioned compounds with groups suitable for linking without interfering with receptor binding can also be used.

It has been found that the combination of the Mtx moiety containing CID with DHFR binding domain containing fusion protein is a highly useful and widely applicable. Mtx and the DHFR receptor present a particularly attractive chemical handle/receptor pair. In addition to having a picomolar binding affinity, the complex of an Mtx moiety and the DHFR binding domain is extremely well characterized. The excellent pharmacokinetic properties of Mtx make it an ideal moiety to be used in procedures where ease of importation into cells is required.

Linking H1 and H2 through a Linker

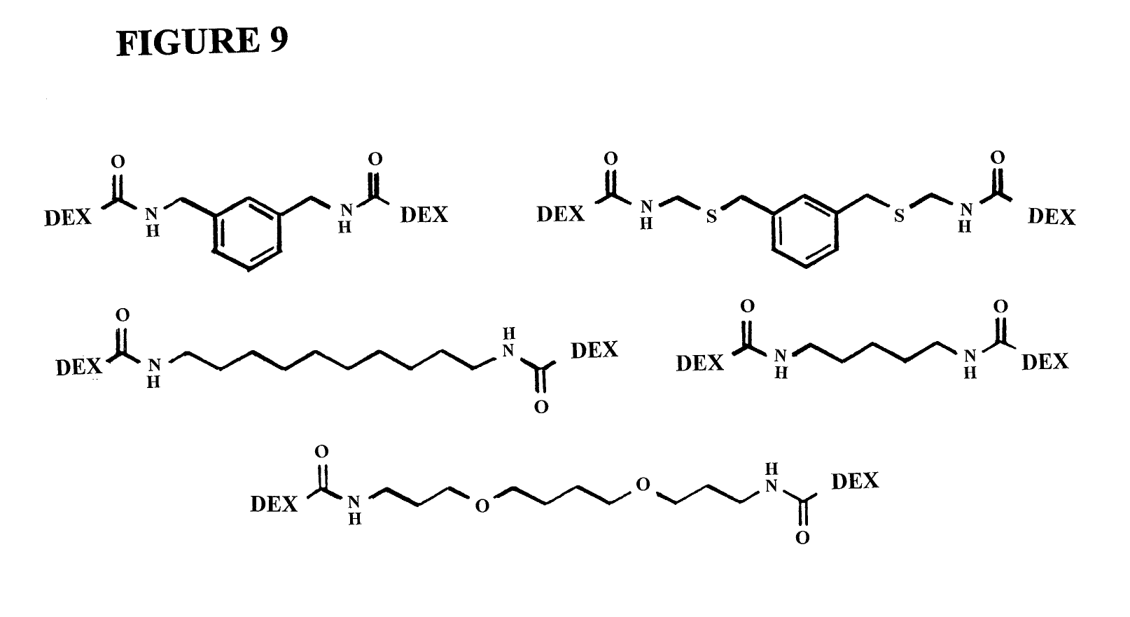

To illustrate how the handles H1 and H2 may be linked together, several of the DEX-DEX compounds that have been synthesized to date are shown in FIG. 9. The linkers are all commercially available or can be prepared in a single step. The linkers vary in hydrophobicity, length, and flexibility. a series of DEX-DEX molecules have been synthesized (FIG. 9). The DEX-DEX molecules shown in FIG. 9 were prepared from Dexamethasone and the corresponding diamines. The C₂₀ a-hydroxy ketone of dexamethasone was oxidized using sodium periodate to the corresponding carboxylic acid in quantitative yield as described. The diamines are commercially available. The diamine corresponding to DEX-DEX 2 was prepared from a,a′-dibromo-m-xylene and aminoethanethiol and used crude. The diamines were coupled to the carboxylic acid derivative of dexamethasone using the peptide-coupling reagent PyBOP under standard conditions in 60-80% yield.

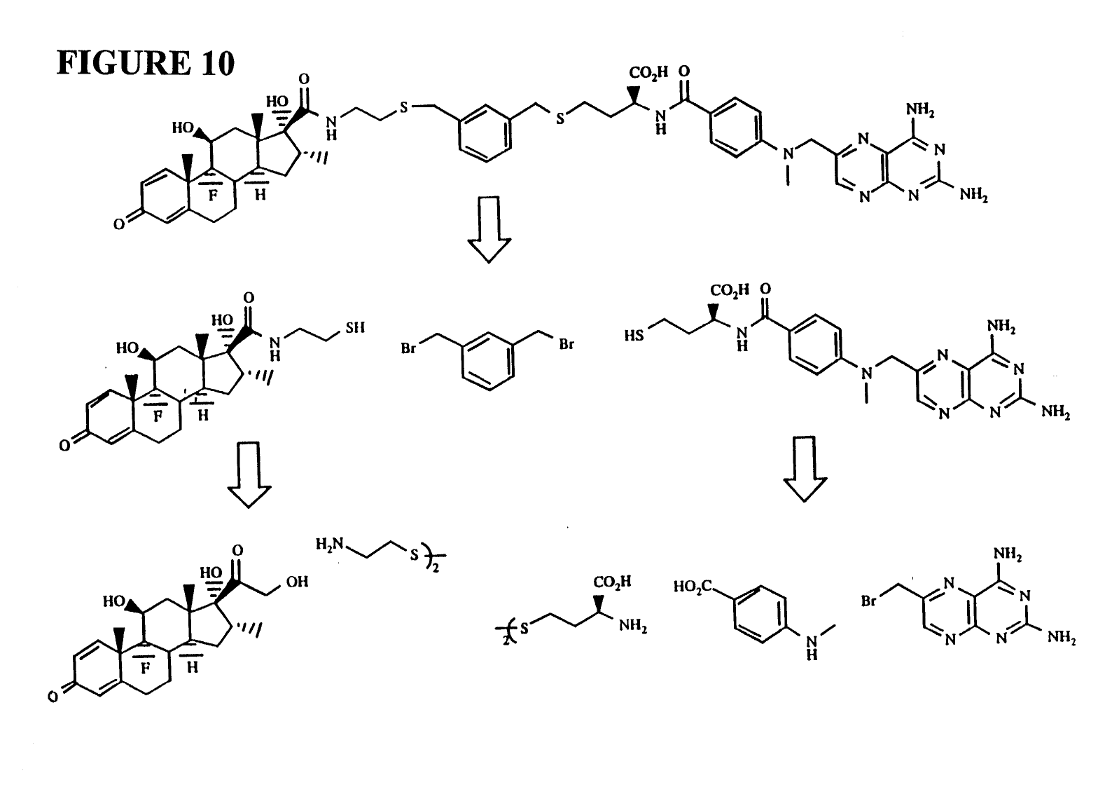

We have synthesized a DEX-MTX molecule. The retrosynthesis is shown in FIG. 10. The synthesis is designed to be modular so that we can easily bring in a variety of linkers in one of the final steps as the dibromo- or diiodo-derivatives. For synthetic ease, the glutamate residue has been replaced with homocysteine. This replacement should be neutral because there is both biochemical and structural evidence that the g-carboxylate of methotrexate can be modified without disrupting DHFR binding. The final compound has been synthesized in 12 steps in 1.3% overall yield. Also synthesized are analogous compounds where the a,a′-dibromo-m-xylene linker is replaced with 1,5-diiodopentane or 1,10-diiododecane. A similar route is used to prepare MTX-MTX molecules.

Design of the Protein Chimeras

The second important feature is the design of the protein chimeras. The yeast two-hybrid assay was chosen in the examples because of its flexibility. Specifically, the Brent two-hybrid system is used, which uses LexA as the DNA-binding domain and B42 as the transcription activation domain. The Brent system is one of the two most commonly used yeast two-hybrid systems. An advantage of the Brent system is that it does not rely on Gal4 allowing use of the regulatable Gal promoter. lacZ under control of 4 tandem LexA operators are used as the reporter gene. Initially, we chose to make simple LexA-GR and DHFR and B42-GR and DHFR fusion proteins that do not depart from the design of the Brent system. In the Brent system, the full length LexA protein which includes both the N-terminal DNA-binding domain and the C-terminal dimerization domain is used. The B42 domain is a monomer. The C-terminal hormone-binding domain of the rat Glucocorticoid Receptor is chosen because this domain was shown to work previously in the yeast three-hybrid system reported by Licitra, et al. Both the E. coli and the murine DHFRs are used because these are two of the most well characterized DHFRs. The E. coli protein has the advantage that methotrexate binding is independent of NADPH binding.

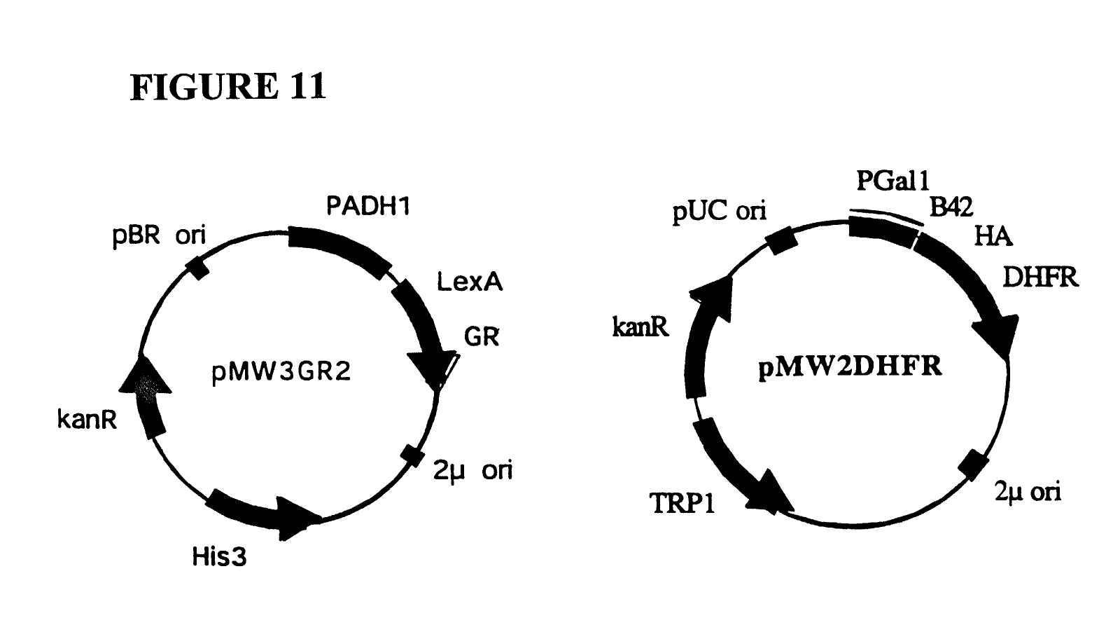

Construction of the LexA- and B42-receptor fusions is facilitated by the availability of commercial vectors for the Brent two-hybrid system. These vectors are shuttle vectors that can be manipulated both in bacteria and yeast. The LexA chimera is under control of the strong, constitutive alcohol dehydrogenase promoter. The B42 chimera is under control of the strong, regulatable galactose promoter. Both the GR and the two DHFR genes were introduced into the multiple cloning sites of the commercial LexA and B42 expression vectors using standard molecular biology techniques. The GR fusions are shown in FIG. 11. The available restriction sites result in a three amino acid spacer between the two proteins in both the GR and the DHFR constructs. The plasmids encoding the LexA- and B42-fusion proteins were introduced in all necessary combinations into S. cerevisiae strain FY250 containing a plasmid encoding the lacZ reporter plasmid.

Three initial assays are conducted: (1) toxicity of the ligand and receptor, (2) cell permeability of the H1-H2 molecules as judged by competition in the yeast three-hybrid system, and (3) activation of lacZ transcription by the H1-H2 molecule as judged by X-gal hydrolysis. All of these experiments have been done as plate assays. The toxicity of the ligand and receptor is judged simply by seeing if either induction of the receptor fusions or application of the ligand to the plate impairs cell growth. Cell permeability is assessed based on the ability of an excess of DEX-DEX or DEX-MTX to disrupt DEX-FK506 induction of lacZ transcription in the yeast three-hybrid system. An excess of DEX-DEX or DEX-MTX should bind to all of the available LexA-GR chimera and disrupt transcription activation so long as the molecule is cell permeable and retains the ability to bind to GR. Effective protein dimerization by H1-H2 is assayed by activation of lacZ transcription.

The DEX-DEX molecules is tested by all three assays. Preliminary results show that neither DEX nor GR are toxic. Under the conditions tried thus far, none of the DEX-DEX molecules tested are efficient at protein dimerization as judged by the lacZ transcription assay. We have been able to repeat the yeast three-hybrid result—activation of lacZ transcription using DEX-FK506, in our lab. DEX-DEX 1 and DEX-DEX 5 have been assayed for cell permeability. At 1 μM DEX-FK506 and 10 μM DEX-DEX, DEX-DEX 1, but not DEX-DEX 5, decreases lacZ transcription in the yeast three-hybrid system by 50%. These results show that a DEX-DEX molecule is cell permeable and retains the ability to bind to GR.

The protein chimeras are varied in four ways: (1) invert the orientation of the B42 activation domain and the receptor; (2) introduce tandem repeats of the receptor; (3) introduce (GlyGlySer)ₙ linkers between the protein domains; (4) vary the DNA-binding domain and the transcription activation domain. We expect these experiments to be carried out over the next two years. The motivation for these experiments is that many different protein fusions have been reported in the literature and these types of modifications have been shown to be critical in these previous experiments. We have designed each of these experiments so that multiple variations can be made simultaneously. Inverting the orientation so that the receptor, not B42, is N-terminal is trivial. We will construct a generic vector that can be used with different receptors. Likewise, since several different DNA-binding domains and activation domains have been used with the yeast two-hybrid system, it is not difficult to vary these domains.

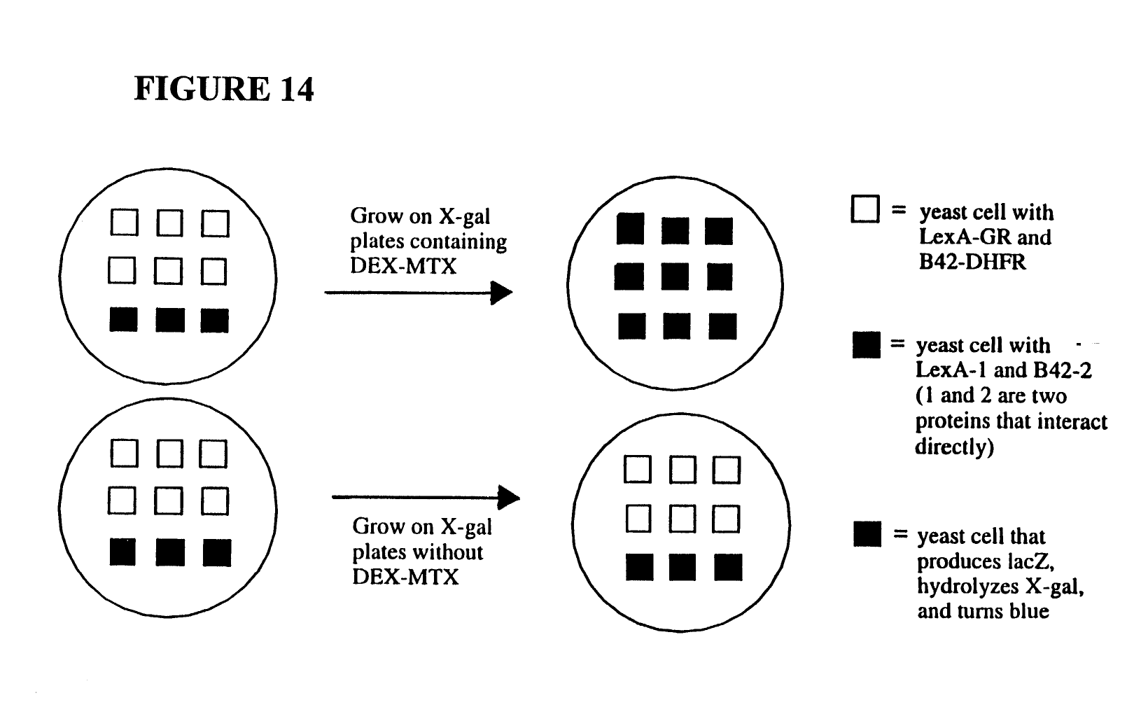

An approach to introducing tandem repeats of the receptor and (GlyGlySer) n linkers that allows us to make multiple constructs simultaneously is provided. As illustrated for GR, the approach to making tandem repeats of the receptor is to use restriction enzymes with compatible cohesive ends (FIG. 14). The same PCR product can then be used to introduce each receptor unit. By including a BamHI restriction site immediately 5′ to the gene encoding GR, a series of (GlyGlySer)ₙ linkers can be introduced essentially as described. This approach relies on the fact that the BamHI site, GGA-TCC, encodes Gly-Ser. This combined approach will allow for the construction of multiple protein chimeras simultaneously. Since a lacZ screen us used, all of these constructs can be assayed simultaneously.

Design of Reporter Genes

A reporter gene assay measures the activity of a gene's promoter. It takes advantage of molecular biology techniques, which allow one to put heterologous genes under the control of a mammalian cell (Gorman, C. M. et al., Mol. Cell Biol. 2: 1044-1051 (1982); Alam, J. And Cook, J. L., Anal. Biochem. 188: 245-254, (1990)). Activation of the promoter induces the reporter gene as well as or instead of the endogenous gene. By design the reporter gene codes for a protein that can easily be detected and measured. Commonly it is an enzyme that converts a commercially available substrate into a product. This conversion is conveniently followed by either chromatography or direct optical measurement and allows for the quantification of the amount of enzyme produced.

Reporter genes are commercially available on a variety of plasmids for the study of gene regulation in a large variety of organisms (Alam and Cook, supra). Promoters of interest can be inserted into multiple cloning sites provided for this purpose in front of the reporter gene on the plasmid (Rosenthal, N., Methods Enzymo. 152: 704-720 (1987); Shiau, A. and Smith, J. M., Gene 67: 295-299 (1988)). Standard techniques are used to introduce these genes into a cell type or whole organism (e.g., as described in Sambrook, J., Fritsch, E. F. and Maniatis, T. Expression of cloned genes in cultured mammalian cells. In: Molecular Cloning, edited by Nolan, C. New York: Cold Spring Harbor Laboratory Press, 1989). Resistance markers provided on the plasmid can then be used to select for successfully transfected cells.

Ease of use and the large signal amplification make this technique increasingly popular in the study of gene regulation. Every step in the cascade DNA→RNA→Enzyme→Product→Signal amplifies the next one in the sequence. The further down in the cascade one measures, the more signal one obtains.

In an ideal reporter gene assay, the reporter gene under the control of the promoter of interest is transfected into cells, either transiently or stably. Receptor activation leads to a change in enzyme levels via transcriptional and translational events. The amount of enzyme present can be measured via its enzymatic action on a substrate.

Host Cell

The host cell for the foregoing screen may be any cell capable of expressing the protein or cDNA library of proteins to be screened. Some suitable host cells have been found to be yeast cells, Saccharomyces cerevisiae, and E. coli.

This invention will be better understood from the Experimental Details which follow. However, one skilled in the art will readily appreciate that the specific methods and results discussed are merely illustrative of the invention as described more fully in the claims which follow thereafter.

Experimental Details

### EXAMPLE 1

We have shown that Dex-Mtx can dimerize a LexA-DHFR and a B42-rGR protein chimera in vivo (Table I). (Lin, 1999) Dex-Mtx was assayed using both plate and liquid assays at extracellular concentrations of 1-100 μM. No activation was observed at concentrations ≦0.1 μM. 100 μM is the limit of Dex-Mtx solubility. Control experiments established that lacZ transcription is dependent on Dex-Mtx. There are only background levels of lacZ transcription when Dex-Mtx is omitted, LexA-DHFR is replaced with LexA, or B42-GR is replaced with B42. Likewise, a 10-fold excess of Mtx competes out Dex-Mtx-dependent lacZ transcription. Interestingly, of the 10 protein chimera combinations tested, Dex-Mtx could only activate lacZ transcription in the context of the LexA-eDHFR and the B42-(Gly6)-rGR chimeras (Table 1). None of the 9 other protein combinations tested worked. This result is consistent with our view that the Dex-Mtx systems (and other dimerization systems) could be further improved both by biochemical and structural characterization and by variation of the protein chimeras and the reporter.

TABLE I
Effect of DEX-Mtx on Dimerization of
Different LexA- and B42-Protein Fusions

| | | | Dex-Mtx |
|---|---|---|---|
| Strain^{a} | LexA Chimera | B42 Chimera | Dimerization^{b} |
| 1 | LexA-eDHFR^{c} | B42-Gly₆^{d}-GR2^{e} | Yes |
| 2 | LexA-eDHFR | B42-rGR2 | No |
| 3 | LexA-eDHFR | B42-(rGR2)₃ | No |
| 4 | LexA-mDHFR^{f} | B42-Gly₆-rGR2 | No |
| 5 | LexA-mDHFR | B42-rGR2 | No |
| 6 | LexA-mDHFR | B42-(rGR2)₃ | No |
| 7 | LexA-rGR2 | B42-eDHFR | No |
| 8 | LexA-rGR2 | B42-mDHFR | No |
| 9 | LexA-(rGR2)₃ | B42-eDHFR | No |
| 10 | LexA-(rGR2)₃ | B42-mDHFR | No |

^{a}S. Cerevisiae strain FY250 containing pMW106 (the lacZ reporter plasmid), pMW103 (encoding the LexA chimera), and pMW012 (encoding the B42 chimera).
^{b}Dex-Mtx-dependent dimerization was determined using standard assays for lacZ transcription. See the text for details.
^{c}the E. coli DHFR.
^{d}In some constructs a 6 Glycine linker was added between B42 and the rGR.
^{e}A mutant form of the hormone-binding domain of the glucocorticoid receptor (residues 524-795, Phe⁶²⁰ →Ser, Cys⁶⁵⁶ →Gly) with increased affinity for Dex was used in these studies.
^{f}the murine DHFR.

### EXAMPLE 2

Cephalosporin Hydrolysis by the 908R Cephalosporinase in the Yeast Three-Hybrid System

The subject invention is exemplified using the components of the yeast three-hybrid system (Licitra, represented in FIG. 2). In this system DEX-FK506 (exemplifying H1-H2) mediates dimerization of the protein fusions LexA-GR (representing reporter V-H1 receptor) and B42-FKBP12 (representing reporter W-H2 receptor) thus activating transcription of a lacZ reporter gene. The chemical handles H1 and H2 and the protein dimerization assay, however, all can be varied.

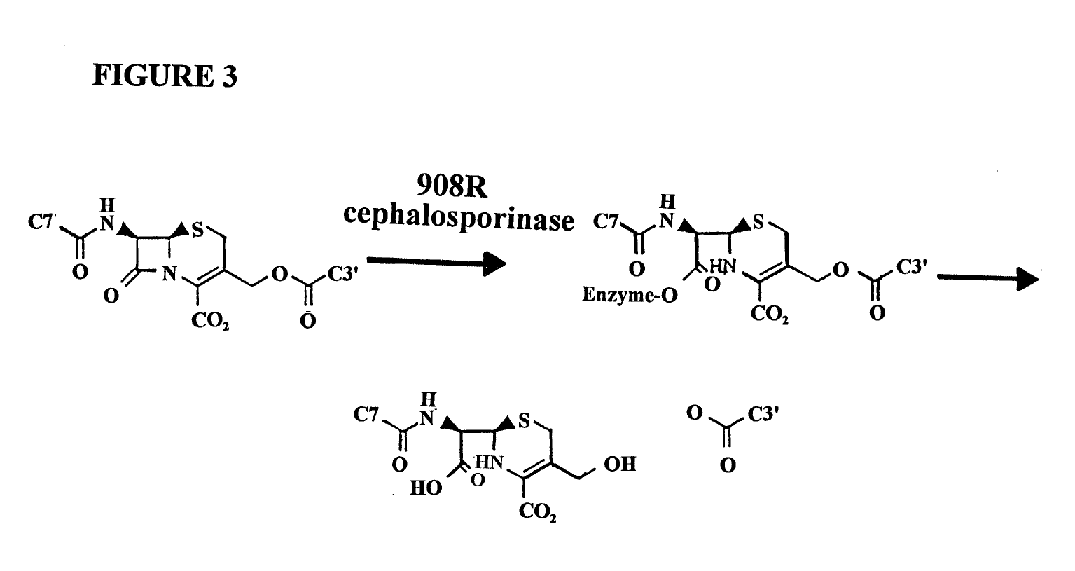

In the subject invention, however, the yeast three-hybrid system is altered by inserting a BOND, B, as well as any required spacers X and Y, so as to form a small molecule having the structure H1-X-B-Y-H2. While there is ample precedent for small-molecule mediated protein dimerization, what remains is to show these assays can be used to select for catalysts. Cephalosporin hydrolysis by a cephalosporinase provides a simple cleavage reaction to demonstrate the selection (FIG. 3). The BOND, B in this example is cephem linkage susceptible to attack by cephalosporinase, such that hydrolysis of the cephalosporinase results in separation of the proteins and deactivation of the transcription of lacZ.

The E. cloacae 908R cephalosporinase is well characterized both biochemically (Galleni; Galleni; Galleni; Monnaie) and structurally (Lobkovsky) and is simple to manipulate. Several approaches have been developed for modifying cephalosporin antibiotics at the C7′ and C3′ positions to improve their pharmacokinetic properties and to prepare pro-drugs. (Druckheimer; Albrecht; Vrudhula; Meyer) Cephalosporin hydrolysis by the cephalosporinase can disrupt protein dimerization and hence be used to discriminate between cells containing active and inactive enzyme. Specifically, (1) (C.)DEX-CEPHEM-(C3′)FK506 is synthesized; (2) DEX-CEPHEM-FK506 is shown to dimerize LexA-GR and B42-FKBP12 and both DEX and FK506 is shown to disrupt the dimerization; (3) induction of the wild type cephalosporinase, but not an inactive Ser⁶⁴ variant, is shown to disrupt cephem-mediated protein dimerization; and (4) cells containing active cephalosporinase are identified based on loss of protein dimerization in a mock screen. A screen for loss of lacZ transcription is sufficient for the screen.

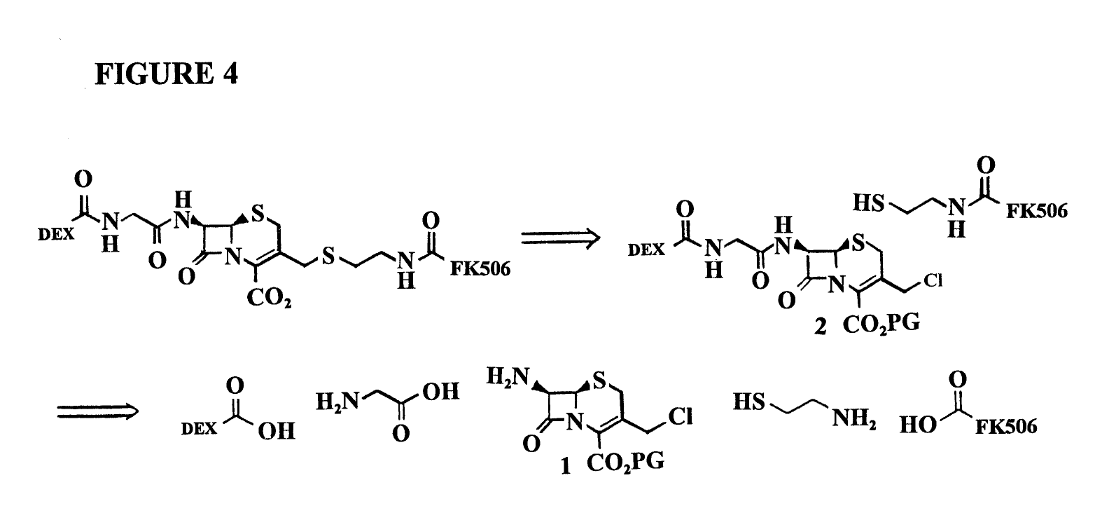

The retro-synthesis of DEX-CEPHEM-FK506 is shown in FIG. 4; it allows H1, H2, and the linker molecules to be varied. The allelic chloride intermediate 2 has been synthesized from cephem 1 in 20% yield in four steps. Mild conditions for coupling H2-SH to the allelic chloride 2 using sodium iodide have been developed; DEX-SH can be coupled in 82% yield. 908R cephalosporinase variants have been constructed both with and without nuclear-localization sequences under control of GALL and MET25 promoters. All of these variants are known to be active in vivo by using the chromogenic substrate nitrocefin, (Pluckthun). Several S. cerevisiae strains suitable for this model reaction have been constructed. DEX-FK506 is know to dimerize LexA-rGR and B42-FKBP12 in these strain backgrounds (yeast three-hybrid system).

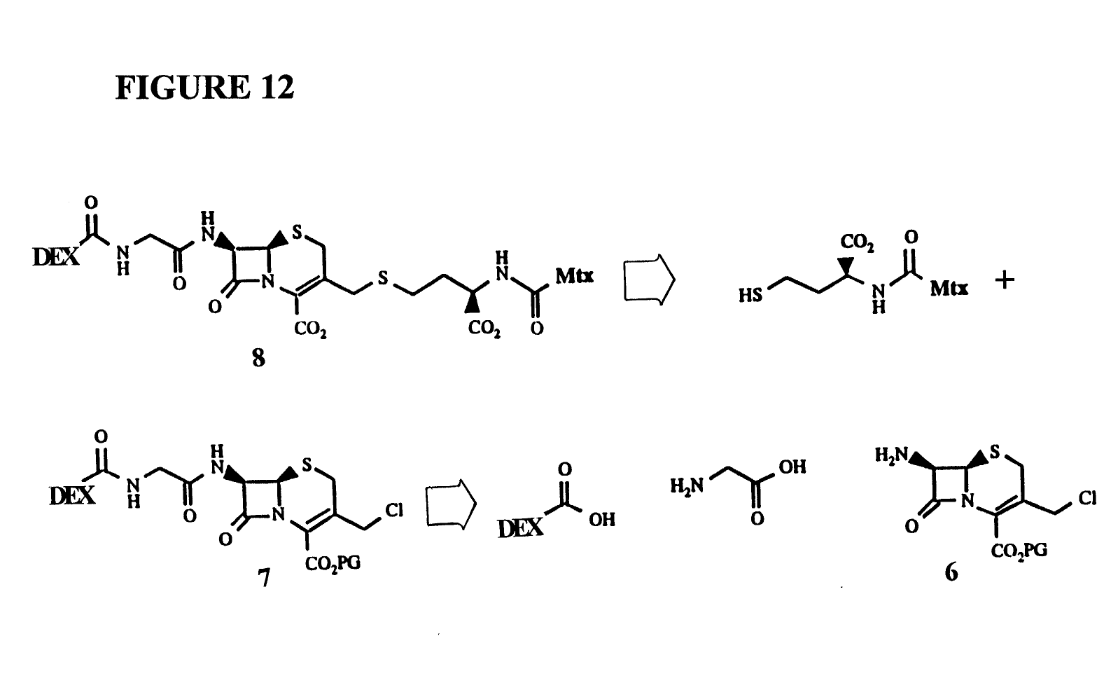

All of the components needed for the proof of principle have been prepared. Specifically, we have developed a modular synthesis of Dex-cephem-Mtx and constructed a S. cerevisiae strain suitable for the proof principle. The retro-synthesis of Dex-cephem-Dex is shown in FIG. 12; it allows H1, H2, and the linker molecules to be varied to optimize the cephem substrate. We have synthesized the allylic chloride intermediate 2 from cephem 1 in 20% yield in four steps. We have developed mild conditions for coupling H2-SH to the allylic chloride 2 using sodium iodide; Dex-SH can be coupled in 82% yield. We have constructed strain FY250/pMW106/pMW2rGR2/pMW3FKBP12 and shown that Dex-FK506 can still mediate dimerization of LexA-rGR and B42-FKBP12 in this strain. The strain provides an additional marker for the enzyme, grows well on galactose and raffinose, and replaces all of the amp^{R} markers with kan^{R} or spec^{R} markers. In addition, we have constructed several constructs for the galactose- or methionine-regulated overexpression of the cephalosporinase. Based on hydrolysis of the chromogenic substrate nitrocefin, (Pluckthun, 1987) we have shown that the cephalosporinase is active in the FY250 background.

The basis for catalysis by the cephalosporinase is studied using combinatorial techniques. Understanding the mechanism is important for anticipating future routes to antibiotic resistance and for developing new cephalosporin antibiotics.

Dex-cephem-Mtx Induces Protein Dimerization in Vivo

Preparation of a Dex-cephem-Mtx (cleavable cephem linker) The cephem substrates were designed such that introduction of the Dex and Mtx ligands would not interfere with cephalosporinase hydrolysis of the cephem core and so that a variety of Dex-cephem-Mtx substrates could be synthesized readily from commercially available materials. (The chemistry of the b-lactams; Durckheimer; Albrecht; Meyer; Zlokarnik) We synthesized four potential Dex-cephem-Mtx substrates from a commercial amino- chloro- cephem intermediate. Dexamethasone was coupled to the C7 amino group of the cephem core via aminocarboxylic acids of different lengths, and methotrexate to the C3′ chloro group via aminothiols of different lengths. All four compounds were prepared from three components in 3-4 steps in 10-30% overall yield.

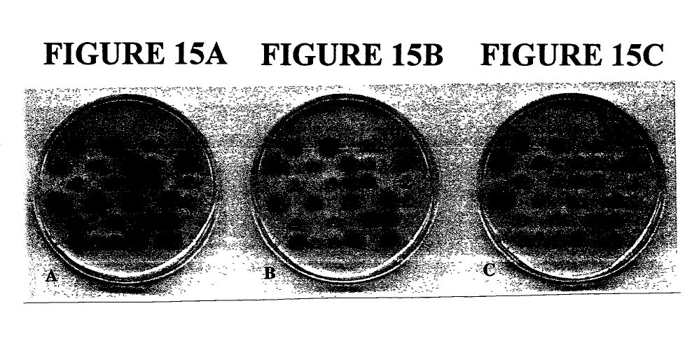

The critical issue was whether introduction of the cephem linker would impede either the cell permeability or the dimerization activity of the Dex-Mtx CID. We screened all four Dex-cephem-Mtx compounds using the yeast two-hybrid lacZ transcription assay and determined that all four compounds are cell permeable and that two of these compounds are capable of inducing protein dimerization in vivo, as shown in FIG. 15. Based on these results, it appears that the length of the linkers between the cephem core and the Dex and Mtx ligands are important; the cephem core must not be too close to the receptor or it will prevent access to the receptor. These results support the general feasibility of preparing CIDs with cleavable linkers and using these compounds in viva with the catalysis screen.

The ability of this Dex-cephem-MTX CID to serve as a read-out for catalysis is evaluated using the well-studied enzymatic reaction, cephem hydrolysis by a cephalosporinase. Hydrolysis of the lactam bond results in expulsion of the leaving group at the C3′ position, effectively breaking the bond between Dex and Mtx.

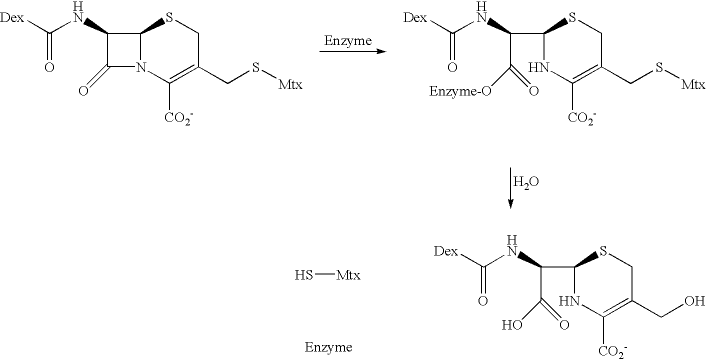

Having identified Dex-cephem-Mtx substrates that are efficient dimerizers in the yeast two-hybrid assay, the next step is to demonstrate that the screen can discriminate between active and inactive enzymes. The penicillin-binding protein (PBP) from Streptomyces R61 provides a good control “inactive” enzyme to compare to the active Q908R cephalosporinase. (Kelly; Ghuysen) Cephalosporinases are believed to have evolved from PBPs.(Ghuysen; Knox) Both enzymes have the same three-dimensional fold and follow the same catalytic mechanism involving an acyl-enzyme intermediate. (Kelly, Lobkovsky) PBPs bind to cephems with high affinity, form the acyl-enzyme intermediate rapidly, but hydrolyze the acyl-enzyme intermediate much more slowly than do Cephalosporinases. We have introduced both the Q908R cephalosporinase and the R61 PBP into yeast shuttle vectors that place the enzymes under control of either a galactose-inducible or a methionine-repressible promoter. Based on plate assays using the chromagenic substrate nitrocefin, (Pluckthun) the Q908R enzyme was expressed in an active form in yeast with either promoter. This assay cannot detect PBP activity.

The Dex-cephem-Mtx CID screen distinguish between the cephalosporinase and the PBP. Yeast strains containing the cephalosporinase hydrolyze the cephem linkage rapidly, disrupting lacZ transcription. The PBP, on the other hand, hydrolyze the cephem linkage too slowly to change the levels of lacZ transcription significantly.

Can the CID Screen Detect Catalytic Activity?

Strong support for the feasibility of using CIDs with cleavable linkers to detect catalytic activity is provided by in vivo selections for protease activity based on cleavage of internal protease sites engineered in a variety of proteins, including Gal4. With an active Dex-cephem-Mtx CID in hand, our next step is to find conditions where the CID screen gives an enzyme-dependent signal. We envision two scenarios which should result in an enzyme-dependent signal: (1) overexpression of the enzyme relative to the LexA- and B42-reporter proteins and (2) expression of the enzyme prior to expression of the LexA- and B42-reporter proteins. The Brent Y2H vectors currently employed in the lab will have to be modified to allow for control over the levels and timing of LexA- and B42-expression. As supplied, the Brent vectors have the LexA fusion protein under control of the strong, constitutive alcohol dehydrogenase promoter (PADH) and the B42 fusion protein under control of the strong galactose-inducible promoter (PGAL). Both vectors contain the high-copy yeast 2μ origin of replication. We plan simply to place the LexA fusion protein under control of a galactose-inducible promoter, just like B42. The GAL promoter is the most tightly regulated promoter available in yeast and is induced by galactose and repressed by glucose. It can be fully repressed, and it can direct expression of a range of intermediate protein concentrations by varying the relative percentages of glucose and galactose in the growth media. Thus, with both LexA and B42 under control of Gal promoters, these reporter proteins can be turned off and then on or expressed at intermediate concentrations in concert. If this approach does not work, there are many other ways to tune the sensitivity of the system. The expression of the enzyme, LexA, and B42 can all be controlled using other inducible or constitutive promoters or by integrating LexA and B42 into the chromosome. The lacZ reporter gene can be replaced with other chromagenic reporters or selectable markers. Alternatively, the sensitivity of the system can be tuned by varying the substrate:product ratio by adding both Dex-cephem-Mtx (substrate) and Dex and Mtx (“product”) to the growth media.

Once conditions were found where we can detect enzyme-dependent cleavage of the cephem linker, we carried out a mock screen as a proof-of-principle experiment. Specifically, plasmids encoding the cephalosporinase and the PBP in a ratio of 1:99 will be introduced into a yeast strain carrying the appropriate protein chimera and reporter genes. Cells harboring the cephalosporinase should be white, while those containing the PBP should be blue. Plasmids from these colonies will be isolated and sequenced to confirm the identity of the expressed enzyme.

Level of Catalytic Activity Detected Using the CID Screen

While these experiments will show that the CID screen can detect catalytic activity, they will not show that the screen can be used to amplify enzymes with low levels of catalytic activity. Thus, our next step is to use cephalosporinase mutants with a range of catalytic efficiencies to quantify and then optimize the sensitivity of the system. Many b-lactamase mutants, either found in clinical settings or constructed by site-directed mutagenesis, have been fully characterized kinetically. Known mutants of the Q908R cephase, the E. cloacae P99 cephase (99% identical), and the E. coli K12 AmpC b-lactamase (71% homologous) are available spanning a wide range of k_{cat}, Kₘ, and k_{cat}/Kₘ values (Table II). To accurately gauge the relative activities of the mutants in the CID and amp^{R} screens, we will determine kinetic rate constants for the corresponding Q908R cephase variants with the Dex-cephem-Mtx and ampicillin substrates and nitrocefin as a control. The Q908R cephase variants will be constructed in the E. coli expression vector by site-directed mutagenesis, using a PCR-based method. These proteins will then be purified by nickel-affinity chromatography, and rate constants will be determined by UV spectroscopy, monitoring the disappearance of absorbance due to the b-lactam bond.

After determining the activity of the mutants with Dex-cephem-Mtx and ampicillin in vitro, these same mutants are tested in the CID and ampR screens. In addition to plate and more quantitative liquid lacZ assays, the mutants will be evaluated using a ura3 reporter gene. Ura3, which encodes orotidine-5′-phosphate decarboxylase and is required for uracil biosynthesis, is used routinely as a selectiable marker in yeast. Since large numbers of protein variants need to be screened for the evolution experiments, it will be important to move from a screen to a growth selection. Ura3 has the advantage that it can be used both for positive and negative selections-positive for growth in the absence of uracil and negative for conversion of 5-fluoroorotic acid (5-FOA) to 5-fluorouracil, a toxic byproduct. Cleavage of the cephem bond and disruption of ura3 transcription will be selected for based on growth in the presence of 5-FOA. The advantage to the 5-FOA selection is that the timing of addition of both the Dex-cephem-Mtx substrate and 5-FOA can be controlled. Several other reporter genes, however, have been reported. The mutants are evaluated in E. coli using nitrocefin screens and amp^{R} selections. Mutants with higher activity (k_{cat}/Kₘ) will still show an enzyme-dependent signal (failure to hydrolyze X-gal or growth in the presence of 5-FOA/nitrocefin hydrolysis or resistance to ampicillin), but at some point these assays will not be able to detect the less active mutants. In addition to suggesting what range of activities can be detected with these assays, these experiments may bring surprising results. For example, it may be that detection correlates more strongly with k_{cat} than with K_{M} or k_{cat}/K_{M}. Assuming a dynamic range of >1000, we will proceed with the enzyme evolution experiments. Otherwise, we will focus on optimizing the sensitivity of the screen until we reach this level of sensitivity. The optimization experiments will continue along the same lines as the proof-of-principle experiments, varying the levels and timing of both protein expression and addition of the substrate and product, except they will be carried out with mutant cephases at the limit of detection.

TABLE II
Wild-type and mutant enzymes are shown with their kinetic rate
constants with the chromogenic cephalosporin nitrocefin, as well as
the percentage of wild-type k_{cat}/Kₘ as calculated in that experiment.

| | | | k_{cat}/Kₘ | |
|---|---|---|---|---|
| Enzyme | Kₘ (μM) | k_{cat} (s⁻¹) | (M⁻¹ s⁻¹) | % WT |
| E. cloacae P99 wt | 25 ± 1 | 780 ± 30 | 3.1 × 10⁷ | 100 |
| E. cloacae Q908R wt | 23 ± 1 | 780 ± 30 | 3.4 × 10⁷ | 100 |
| K12 AmpC wt | 500 ± 100 | 490 ± 90 | 1.0 × 10⁶ | 100 |
| P99 286-29O TSFGN | 19 ± 0.5 | 261 ± 7 | 1.37 × 10⁷ | 96 |
| P99 286-290 LTSNR | 43 ± 2 | 330 ± 11 | 7.7 × 10⁶ | 54 |
| P99 286-290 NNAGY | 31 ± 11 | 53 ± 10 | 1.7 × 10⁶ | 12 |
| K12 Y150S | 108 ± 21 | 2.11 ± 0.12 | 1.9 × 10⁴ | ^{˜}1 |
| K12 Y150E | 356 ± 34 | 0.51 ± 0.03 | 1.4 × 10³ | ^{˜}0.1 |
| Q908R S64C | >1000 | >18 | 1.76 × 10⁴ | 0.05 |

### EXAMPLE 3

CIDs can be used to screen CDNA libraries based on biochemical function. This glycosidase example is used to determine the best method for expressing the cDNA clones and to optimize the screening process.

Proof of Principle β-Galactosidase Activity Assays

Table III explains the components of each strain. Each strain was constructed from the parent yeast strain FY250 and also contains the pMW106 plasmid, which has the LacZ reporter gene that is turned on only in when the LexA DNA binding domain and the B42 activation are brought in tot he vicinity of each other. We use several different strains because we use DHFR from two different species, mDHFR is from murine, while eDHFR is from E.coli. We are asl oable to switch the small moleculebinding domains. For example, the strain containing LexA-eDHFR with B42-rGH2 is a different strain and behaves differently from the strain containing LexA-rGR2 with B42-eDHFR. We also put in short 6 amino acid linkers between the two domains of our protein chimeras and thus these are different strain as well.

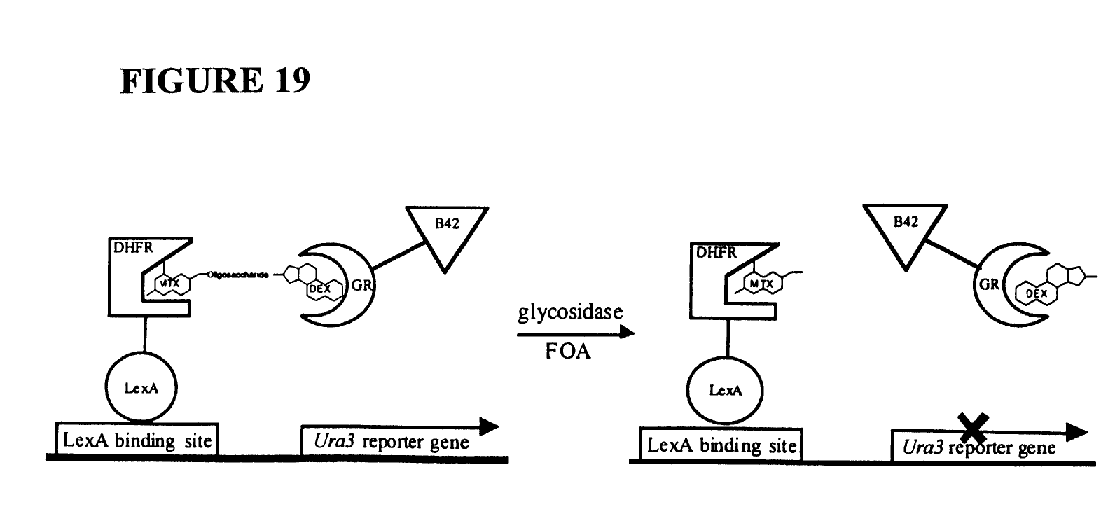

Next, we have chosen to screen a yeast cDNA library for proteins with glycosidase activity (FIG. 19).

Table III. Identification of stains used. (Key: eDHFR=E.coli Dihydrofolate Reductase; rGR2=stereoid binding domain of rat Glucocorticoid Receptor (aa 524-795) with point mutations; (rGR2)3=trimer of rGR2; mDHFR=murineDihydrofolate Reductase; gly6=6 amino acid linker conaining 6 glycines; (GSG)2=6 amino acid linker containing glycine-serine-glycine-glycine-serine-glycine.)

β-Galactosidase Activity Assay Results

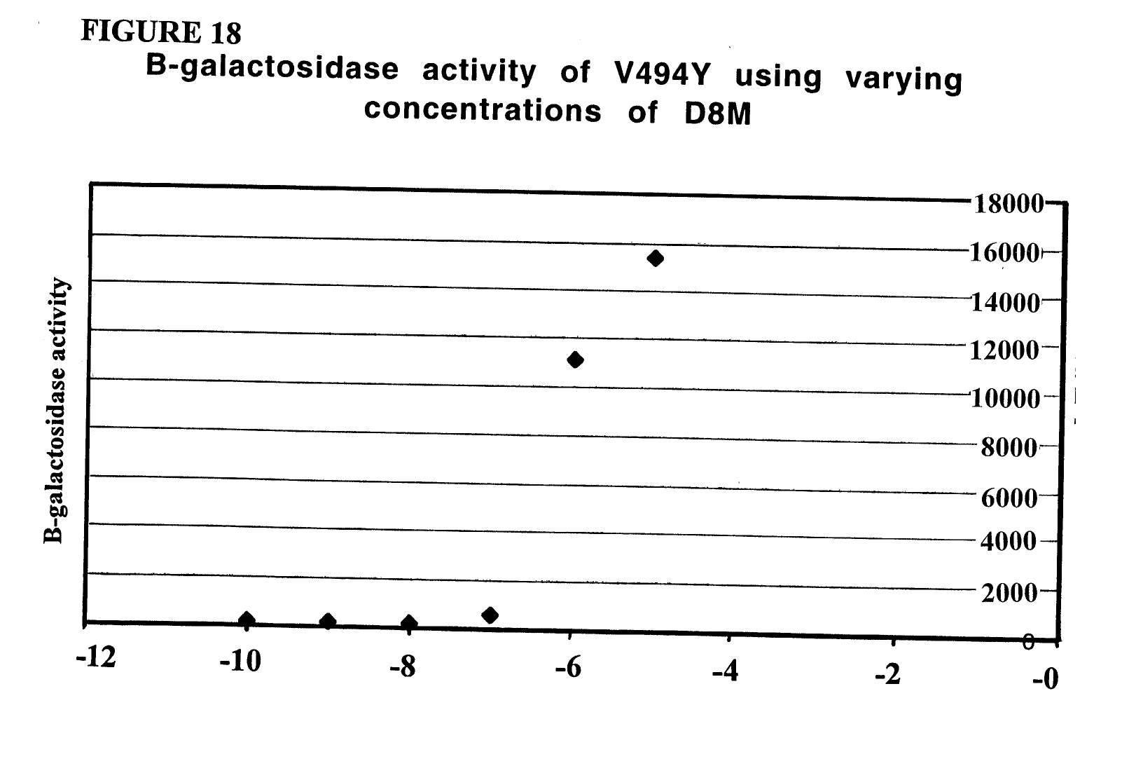

The results in Table IV are averages of two separate trials. Each strain was examined with small molecules and without small molecules. The absolute activity is given as the β-galactisidase activity with small molecule subtracted from the β-galactosidase activity without small molecule. The average β-galactosidse activity for a strain without small molecule (i.e. the negative control) was about 100 β-galactosidase units. V133Y is a positive control and shows β-galactosidase activity regardless of the presence of small molecule. The β-galactosidase activity of strain V494Y using varying concentrations of D8M is shown in FIG. 18.

Table IV—β-galactosidase Activity Assays

Glycoconjugates are the most functionally and structurally diverse molecules in natures.[Varki, 1993] Moreover, it is now well established that carbohydrates and protein- and lipid-bound saccharides play essential roles in many important biological processes, including cell structure, protein targeting, and cell-cell interactions.[Varki, 1993] Accordingly, glycosidases with a broad array of substrate specificities are required to breakdown and modify polysaccharides, glycoproteins, and glycolipids.

Using CIDs with structurally diverse carbohydrate linkers, we screen a S. cerevisiae CDNA library based on glycosidase activity. There are many examples of well-characterized glycosidases identified in other organisms that are yet to be identified in S. cerevisiae. a-Amylase[Sogaard, 1993; Vihinen, 1990; Qian, 1994; Wiegand, 1995; Fujimoto, 1998; Wilcox, 1984] and xylanase[Wong, 1988; Biely, 1997] are endo-glycosidases that break down polysaccharides involved in energy storage and cell structure, respectively. Glycoproteins are synthesized by modification of a core glycoside. The GlcNAcb1®Asn and GlcNAcb1®4GlcNAc linkages in Asn-linked carbohydrates are cleaved by peptide-N⁴- (N-acetyl-b-glucosaminyl) asparagine amidase (PNGase F) and endo-b-N-acetylglucosaminidases (Endo H and Endo F1), respectively. [Tarentino, 1990; Tarentino, 1992; Robbins, 1984; Trimble, 1991] Since each of these enzymes are endo-glycosidases, the CID ligands should not interfere with the enzyme-catalyzed reaction. Likewise, by making a small library of carbohydrate linkers, we screen in an undirected fashion.

The diversity of naturally occuring carbohydrates requires us to make a library of Dex-Mtx CIDs with different carbohydrate linkers. Recent advances in the synthesis of oligosaccharides, both in the coupling methods[Schmidt, 1986; Toshima, 1993; Boons, 1996] and in the solid-phase synthesis,[Danishefsky, 1993; Seeberger, 1998; Yan, 1994; Liang, 1996] make it possible to synthesize these linkers. We have chosen to use a method developed by Kahne and co-workers which uses anomeric sulfoxides as glycosyl donors and synthesizes carbohydrates from the reducing to the non-reducing end.[Yan, 1994; Liang, 1996] This method can be used both in solution and on solid-support, can form both a- and b-glycosidic bonds, and utilizes readily-synthesized intermediates. Several alternative methods, however, are available, including Wong and co-workers' one-pot solution synthesis[Zhang, 1999; Ye, 2000] and the solid-phase glycal strategy reported by Danishefsky and co-workers.[Danishefsky, 1993; Seeberger, 1998]

We screen a yeast cDNA library based on glycosidase activity using Dex-Mtx CIDs with cleavable glycosidic linkers (FIG. 12). Concurrently, we identify glycosidases from a S. cerevisae cDNA library by screening for cleavage of CIDs with glycosidic linkages. The Dex-Mtx yeast two-hybrid assay is used as the screen by replacing Dex-Mtx with Dex-oligosaccharide-Mtx. First, we carry out a control where we screen for a known glycosidase, chitinase, using a defined substrate. Second, we screen for unknown glycosidases by using a small library of substrates with different glycosidic bonds.

Screen of a S. cerevisiae cDNA Library Based on Glycosidase Activity

Using Dex-Mtx CIDs with cleavable oligosaccharide linkers, we screen a S. cerevisiae cDNA library based on glycosidase activity. As a control, we screen for a known S. cerevisiae glycosidase, chitinase. Then, we synthesize a small library of Dex-carbohydrate-Mtx substrates and screen the S. cerevisae cDNA library to identify glycosidases from the >3000 ORFs of unkown function in S. cerevisiae.

Introduction of a S. cerevisiae cDNA Library into the CID Selection Strain

The first step of both the chitinase control and the random oligosaccharide library is to introduce a S. cerevisiae cDNA library into the CID selection strain. We use a cDNA library reported by Fields and co-workers. [Martzen, 1999] In this library, each cDNA clone is expressed as a GST-fusion protein under control of a copper-inducible promoter on a shuttle vector with a leu2 marker.[Martzen, 1999; J. R. Hudson, 1997] Transformation efficiencies in yeast are ca. 10⁶-10⁷ using the lithium acetate method, so there is ample redundancy to screen all 6,000 ORFs in S. cerevisiae. Active clones can be identified by sequencing the plasmid. For the chitinase control experiment, we make a library with a subset of cDNA clones to test different approaches for expressing the cDNA clones.

Can the S. cerevisiae Chitinase be Identified Using the CID Selection?

We begin by screening a S. cerevisiae cDNA library for a known glycosidase, chitinase. Chitinase hydrolyzes chitin, polymers of b-1,4-linked N-acetylglucosamine (GlcNAc) that play a structural role in the cell.[Muzzarelli, 1977] Chitinases from several organisms, including S. cerevisiae, have been cloned and characterized.[Correat, 1982; Kuranda, 1987; Kuranda, 1991] It is known that this enzyme can hydrolyze oligomers of b-1,4-GlcNAc ranging from trimers to heterogeneous polymers, suggesting that CIDs such as Dex-(GlcNAc) -Mtx should be efficient substrates for this enzyme. Several efficient syntheses of β-1,4-linked GlcNAc have been published.[Banoub, 1992].

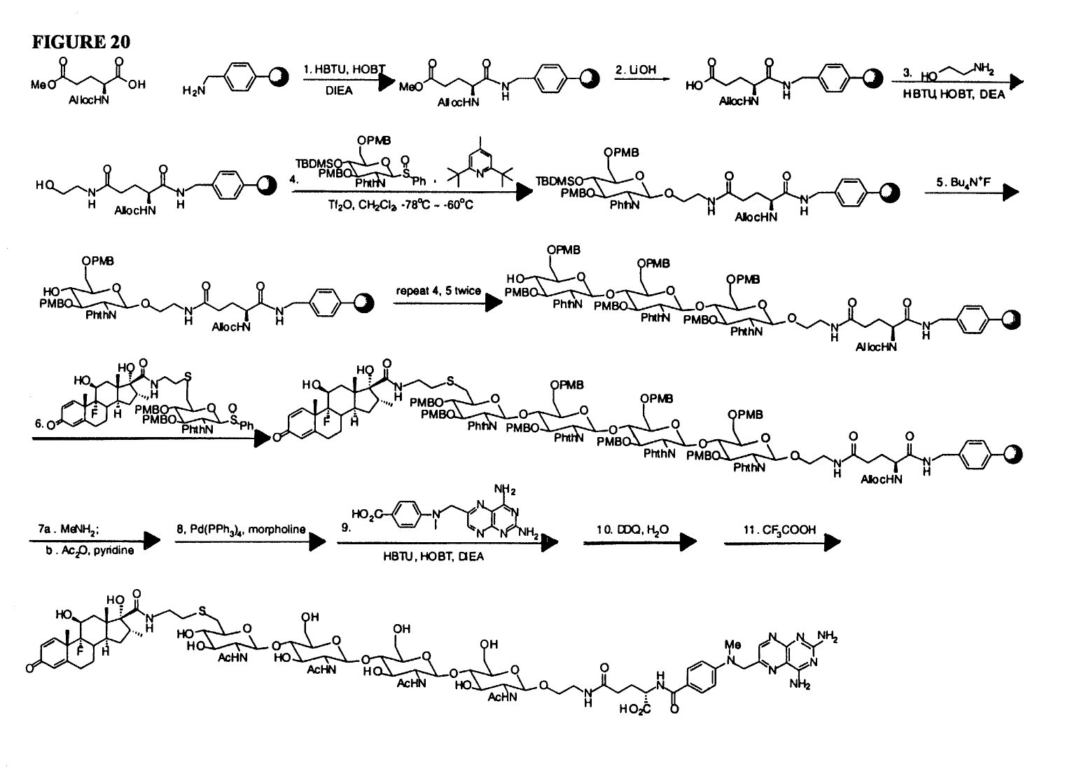

The retro-synthetic analysis of our Dex-(GlcNAc)ₙ-Mtx CID substrate is shown in FIG. 20.

The growing carbohydrate chain is linked to the solid support via the Glu portion of Mtx. The glycosidic linkages are formed essentially as reported by Kahne and co-workers using sulfoxide glycosyl donors.[Yan, 1994; Liang, 1996] The final carbohydrate is introduced as a Dex derivative, and the Mtx synthesis is completed prior to cleavage from the solid support. This synthesis allows the oligosaccharide linker to be varied and both the Dex and the Mtx ligand to be introduced before cleavage from solid support. Alternatively, the synthesis can be carried out in solution,[Kahne, 1989] or other methods for carbohydrate synthesis can be employed.[Zhang, 1999; Ye, 2000; Danishefsky, 1993; Seeberger, 1998 We start with a GlcNAc tetramer as trimers have been shown to be the shortest efficient substrates for chitinases.[Watanabe, 1993].

Initially, lacZ plate assays are used to verify that the Dex-(GlcNAc)ₙ-Mtx substrates are efficient dimerizers in the yeast three-hybrid assay. The results with Dex-cephem-Mtx support the feasibility of incorporating structurally diverse linkers into the CIDs. If the initial chitinase substrates, however, are not efficient dimerizers, the linkers between the CID ligands and the GlcNAc oligomer can be varied, or alternate dimerization assays can be tested. Since large numbers of cDNA clones need to be screened, the transcriptional read-out of the yeast three-hybrid assay may be changed from a screen to a growth selection. Specifically, ura3, which encodes orotidine-5′-phosphate decarboxylase and is required for uracil biosynthesis, replaced lacZ as the reporter gene. [Boeke, 1984] Ura3 has the advantage that it can be used both for positive and negative selections-positive for growth in the absence of uracil and negative for conversion of 5-fluoroorotic acid (5-FOA) to 5-fluorouracil, a toxic byproduct. Cleavage of the glycosidic bond and disruption of ura3 transcription is selected for based on growth in the presence of 5-FOA. The advantage to the 5-FOA selection is that the timing of addition of both the Dex-(GlcNAc)ₙ-Mtx substrate and 5-FOA can be controlled. Several other reporter genes, however, can be used.

One problem that has the potential of occurring is that the Dex-(GlcNAc)ₙ-Mtx substrate becomes unstable either because of its intrinsic half-life in water or because it is turned over by cellular glycosidases. However, if the substrate has a short half-life in water, the assay conditions can be modified so that the substrate is added late in the assay after the cells have grown to a high density, the substrate can be continuously replenished, or the pH of the media can be buffered. Turnover by cellular glycosidases can simply be seen as an assay in and of itself. Using traditional genetic approaches, random mutations can be introduced into the S. cerevisiae genome or the tagged knock-out strains of Winzeler et al. can be used.[Winzeler, 1999] Cells containing a disruptive mutation in the gene or genes cleaving the Dex-(GlcNAc)ₙ-Mtx substrate can be selected for by growth in the absence of uracil.

The final step is to use the Dex-(GlcNAc)ₙ-Mtx substrate to pull out chitinase from a S. cerevisiae cDNA library. As described above, a 5-FOA growth selection is used to screen the Fields cDNA library. In the absence of chitinase, Dex-(GlcNAc)ₙ-Mtx induces ura3 transcription, and 5-FOA is converted to the toxic byproduct 5-fluorouracil. Thus, only cells containing active chitinase, or another enzyme that can cleave the substrate, survive. The cDNA clone is readily identified by isolating the plasmid, sequencing the N-terminus of the clone, and comparing this sequence to that of the S. cerevisiae genome. The advantage of using a known enzyme is that the enzyme can be tested independently or used to spike the cDNA library. The enzyme can be purified, and the Dex-(GlcNAc)ₙ-Mtx substrate can be tested in vitro. We can vary the format of the cDNA library, the Dex-(GlcNAc)ₙ-Mtx substrate, the screen, or the assay conditions, or even use a different glycosidase as a control.

Can Glycosidases be Identified from the >3000 Unassigned ORFs in S. cerevisiae Using the CID Selection?

The next step is to determine the activity of the >3000 ORFs in S. cerevisiae with unknown function. To detect glycosidase activity, the screen is run exactly as with the chitinase control except using Dex-oligosaccharide-Mtx substrates with different glycosidic linkages. The glycosidic linkages is based on the types of carbohydrates and glycoconjugates naturally occuring in yeast. Several activities, including amylase, [Sogaard, 1993; Vihinen, 1990; Qian, 1994; Wiegand, 1995; Fujimoto, 1998; Wilcox, 1984] xylanase,[Wong, 1988; Biely, 1997; Georis, 1999] and endo-N-acetylglucosamine hydrolysis activity,[Tarentino, 1990; Tarentino, 1992; Robbins, 1984; Trimble, 1991] can be targeted specifically.

Dex-Mtx CIDs with different oligosaccharide linkers are prepared using the same strategy as for the chitinase substrate (above). The sulfoxide glycosyl donor method for carbohydrate synthesis allows a variety of sugar monomers to be introduced. [Kahne, 1989] Moreover, both the regio- and stereo-chemistry can be controlled. [Yan, 1994; Liang, 1996] As with the chitinase control, the 5-FOA growth selection is used to identify enzymes that cleave the various glycosidic linkages. Each glycoside subsrate is tested individually. Mixtures of substrates cannot be tested because the uncleaved substrates would continue to activate ura3 transcription. If the screen does not pick up any enzymes, known glycosidases from other organisms may be used as controls both for the growth selections and to test the Dex-Mtx substrates in vitro.

the foregoing permits the characterization of in vitro activity and biological function of glycosidases identified using the CID screen. Similarly, CDNA libraries from other organisms can be screened. The Dex-Mtx substrates can be used to evolve glycosidases with unique specificities. In addition, the cDNA screen can be extended to other classes of enzymes, such as proteases.

### EXAMPLE 4

Development of CID with a Suicide Substrate (“Covalent CID”)

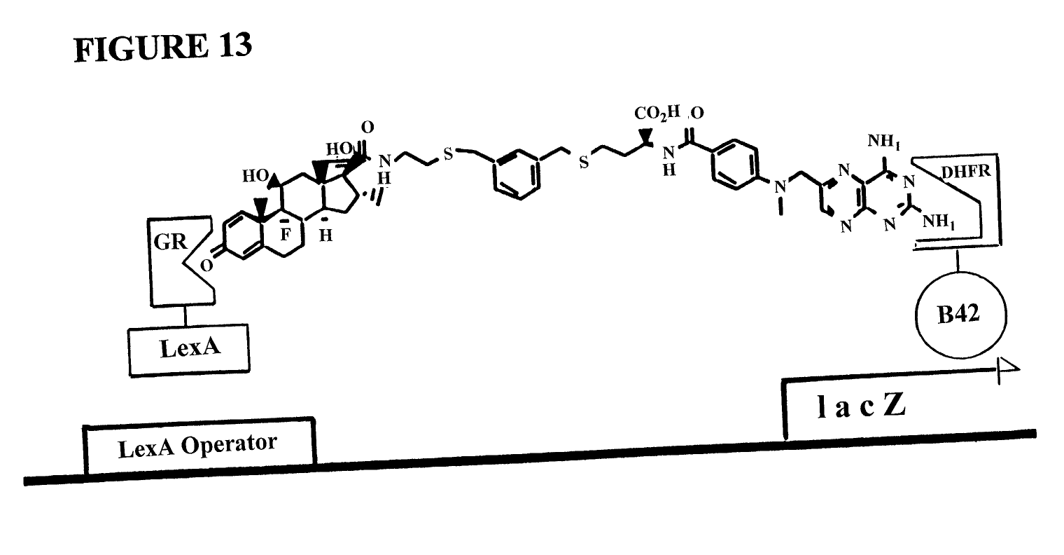

As shown in FIG. 13 and the accompanying discussion, 4 non-covalent interactions have to take place simultaneously for the reporter protein to be activated. Specifically, 1) the DNA-binding protein-DNA interaction, 2) the 1^{st} ligand-receptor interaction, 3) the 2^{nd} ligand-receptor interaction, and 4) the activation domain-transcription machinery interaction.

However, it is possible to replace the 1^{st} ligand-receptor pair (Dex-GR in FIG. 13) with a small molecule-receptor pair that will form an irreversible covalent linkage, making a system with only 3 non-covalent interactions. Such an approach allows for the screening of small molecules to identify their cellular targets. This covalent CID system is used for screening the ligand receptor interaction, which used to be laborious work by using the photo cross linking, radio labeled ligand binding and affinity chromatography techniques. The covalent system is more sensitive than the Dex-Mtx system because the covalent bond gives zero k_{off} for the covalent ligand-protein binding pair and then the cut-off Kd of the whole system is enhanced.

The covalent CID system is constructed by the same principles as other CID, except that one of the ligand-receptor pairs is selected so that in vivo the pair is fixed by a covalent bond and the cell read-out will be depended on the other ligand-receptor interaction.

The covalent CID should find broad use anytime a covalent linkage between the ligand and receptor increases the efficacy of the system. The ligand might be a small molecule, e.g. a drug, and the target may be a protein responsible for the drugs efficacy or for unwanted toxic side effects. The small molecule may also be a cofactor or hormone and the goal might be to screen a genomic library to identify proteins that bind to the given cofactor or hormone. In both cases, the covalent CID allows not only high affinity (nM), but also moderate affinity (μM), interactions to be detected. Reasonable targets for covalent CIDs include suicide substrate-enzyme pairs, which in this example are Fluorouracil-Thymidylate Synthase and Cephen-Penicillin Binding Protein.

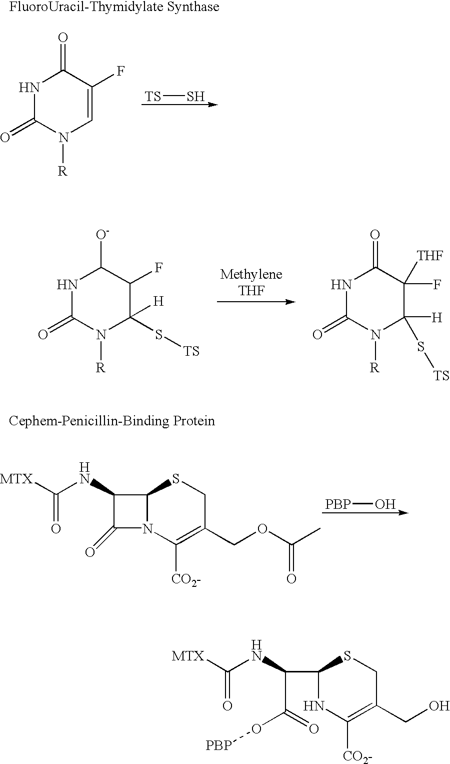

The above two suicide substrate/enzyme pairs are selected because they are stable at physiological pH and activated toward covalent modification only in the enzyme active-site. In addition, an antibiotic-bacterial enzyme pair have the advantage that they can readily be transferred to mammalian cells without toxicity effects. Furthermore, as show in our PCT International Publication No. WO 01/53355, the contents of which are hereby incorporated by reference, Dex-cephem-Mtx CIDs are cell permeable.

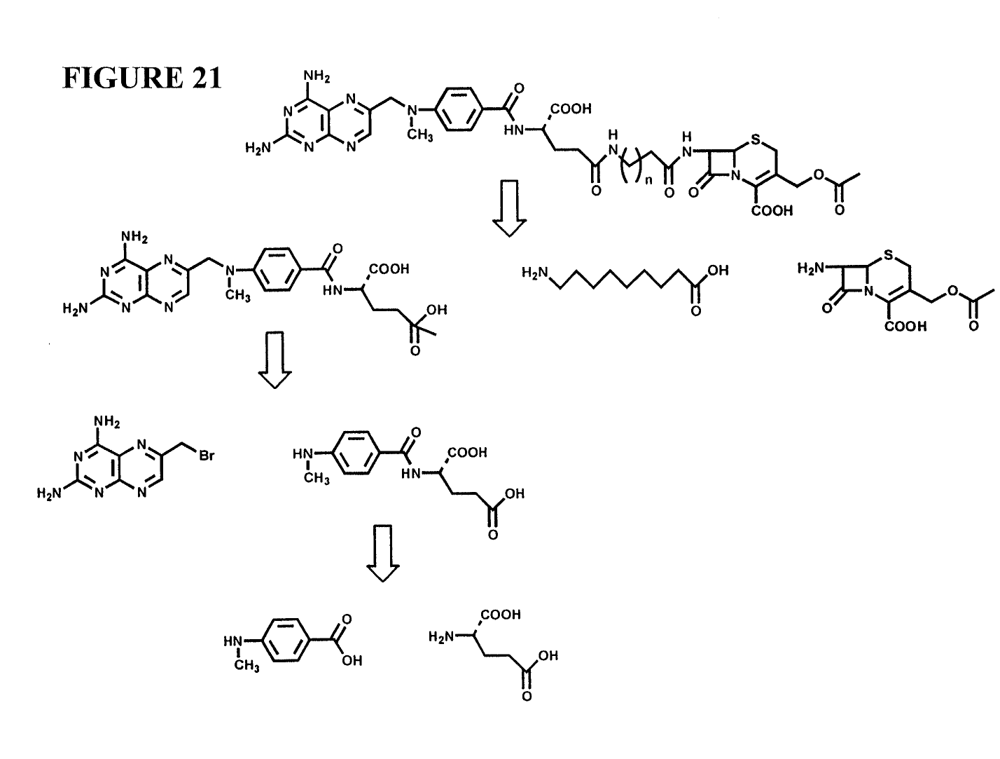

In this example we use cephem and Streptomyces R61 penicillin binding protein to generate this covalent bond. The reaction between β-lactam compounds and the penicillin binding protein is well studied, and the R61 enzyme is well-characterized biochemically and structurally (Kuzin et al, 1995; Kelly, 1995). The synthesis of β-lactam compounds is well established also. The Mtx-eDHFR ligand-small molecule pair is kept is this new system because this pair has higher affinity and better small molecule cell permeability than the Dex-rGR ligand-receptor pair. The cephem-Mtx CID shown below is synthesized by analogy to our syntheses of Dex-cephem-Mtx, as shown in FIG. 21.

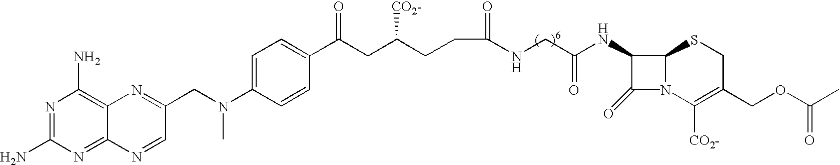

The above CID for this covalent system consists of two ligands: one consists of MTX, which binds to DHFR; the other is cephem, which can covalently bind to R61. These two ligands were connected by a hydrophobic linker. We chose to incorporate the Mtx to the C7 position of the cephem because this position can be modified without disrupting the cephem's activity.

Testing of this or similar molecule for its ability to activate lacZ transcription in the yeast three-hybrid assay when the GR receptor is replaced with the R61 Penicillin-Binding Protein has been described. Since Mtx-DHFR variants with a broad range of Kds and kₒₙ and k_{off} are known, we can use these variants to compare the ability of the noncovalent Dex CID and the covalent cephem CID to detect moderate affinity interactions. The cephem and the Mtx-cephem linker can be readily varied and other suicide substrate-enzyme pairs can be evaluated.

Subcloning of R61 Penicillin Binding Protein; Generation of E. coli. Strain.

The elements of the covalent CID system correspond with the Dex-Mtx yeast three-hybrid system. It is composed of the small molecule, the LexA DNA binding domain chimera (LexA-DHFR or LexA-R61), the B42 transcription activation domain chimera (B42-R61 or B42-DHFR), and the report gene (lacZ). Plasmids of protein chimeras were constructed by subcloning and were transferred to yeast.

Table 1 lists all of the R61 constructs prepared. All of the plasmids were sequenced and no mutation was found.

TABLE 1

| Plasmid on which | | | Strain |
|---|---|---|---|
| construct is based | Fusion protein | Plasmid Name | Name |
| p/T/R61 | R61 without EcoR I | pTEMPPKX720 | V720E |
| pMW102 | B42-R61 | pGBPKT719 | V719E |
| pMW102 | B42-GSGGSG-R61 | pGBPKT779 | V779E |
| pMW103 | LexA-R61 | pALPKH755 | V755E |
| pMW103 | LexA-GSGGSG-R61 | pALPKH754 | V754E |

Yeast Strain

All of the final diploid yeast screening strain will be generated by mating. pMW102 Plasmids were transformed to FY250 and EGY48 strains. pMW103 plasmids and reporter gene plasmids (pMW106 or pMW112) were transformed to FY251 strain. Table 2 lists all of the haploid yeast strains prepared.

TABLE 2

| Haploid strain | Plasmid | Plate | Strain # |
|---|---|---|---|
| FY250:V240Y | pMW102 R61 | HT_03 | NN |
| FY250 | pMW102GSGGSGR61 | HT_03 | NN |
| FY250 | pMW102eDHFR | HT_03 | NN |
| FY250 | pMW102 blank | HT_03 | NN |
| FY250 | pMW102rGR2 | HT_03 | NN |
| EGY48:BTC | pMW102 R61 | HT_04 | NN |
| EGY48 | pMW102GSGGSGR61 | HT_04 | NN |
| EGY48 | pMW102eDHFR | HT_04 | NN |
| EGY48 | pMW102 blank | HT_04 | NN |
| EGY48 | pMW102rGR2 | HT_04 | NN |
| FY251:V525Y | pMW103R61; pMW106 | HT_01 | NN |
| FY251 | pMW103R61; pMW112 | HT_01 | NN |
| FY251 | pMW103eDHFR; pMW106 | HT_02 | NN |
| FY251 | pMW103eDHFR; pMW112 | HT_02 | NN |
| FY251 | pMW103GSGGSGR61; pMW106 | HT_01 | NN |
| FY251 | pMW103GSGGSGR61; pMW112 | HT_01 | NN |
| FY251 | pMW103blank, pMW106 | HT_02 | NN |
| FY251 | pMW103blank, pMW112 | HT_02 | NN |

The haploid strains of are mated, resulting in diploid strains that contain the pMW103, the pMW102 and the reporter plasmid, or some permutation thereof. The yeast strains are phroged to SC(H-U-T-)/galactose/raffinose liquid media in 96-well plates and incubated in a 30° C. shaker for two days and then phroged to X-gal plates, and X-gal plates with 1 mM Mtx-cephem. The plates are incubated at 30° C. for two days. The colonies of cells having the plasmid being selected for (for example: the strain which has R61-LexA fusion protein, DHFR-B42 fusion protein, and the pMW106 reporter gene) turn blue on the Mtx-cephem X-gal plate, but are white on the general X-gal plate. The positive control (yeast two hybrid system) and negative control (lacking one of the fusion proteins) is used during the experiment.

Comparison between the covalent CID system and the Dex-Mtx CID system shows that covalent CID system gives positive results at lower CID concentrations. Any mutation on the DHFR gene that lowers the affinity of the DHFR protein to Mtx results in a negative result in the Dex-Mtx CID system, but a positive result in the covalent CID system.

### EXAMPLE 5

cDNA Binding Screen: Steroids

We can Screen proteins from cDNA libraries based on binding activity using a modified yeast-three hybrid assay. The screening of cDNA libraries method is based on function. The advantage of this method is that it is straightforward using existing technology.

Initially we synthesize several, e.g. 5-10, CID's each comprising a methotrexate moiety covalently linked to a different steroid. These steroid-Mtx CIDs are screened against a S. cerevisiae two-hybrid library where DBD-DHFR is held constant and AD-cDNA library is the variable. Each time a given steroid binds to a given S. cerevisiae protein, the reporter gene should be activated. The steroid-Mtx analogs can be chosen at will, and are their synthesis is known.

First, we test Dexamethasone-Mtx, primarily because Dex has a common A-ring. Second, we synthesize different steroids with common A-ring structures. We have chosen to focus on varying A-rings because, 1) natural steroids often differ primarily in their A-rings, 2) it allows us to use the same chemistry to synthesize all of the steroid-analogs, and 3) there are many examples of natural steroid-receptor complexes where the A-ring is buried in the protein-binding pocket, while the D-ring can be derivatized without disrupting receptor binding. Specifically, we synthesize Steroid-Mtx CIDs based on the steroids Dexamethasone, Estrone, Progesterone, Cholesterol, and Lanosterol. These steroids are chosen because they have representative A-rings and because they play important physiological roles (Lanosterol specifically in yeast):

To simplify the chemistry, steroids that retain similar A/B/C rings, but have one of two D rings, may be used. Specifically, such steroids are 3P-Hydroxy-5-cholen-24-oic acid (Aldrich), Eburicolic acid (Aplin Chemicals), Progesterone (Aldrich), Estrone (Aldrich), and Dexamethasone (Aldrich).

If any steroid is not available as a carboxylic acid, it can be converted to a carboxylic acid by the representative scheme shown in FIG. 19.

These carboxylic acids will then be coupled to methotrexate by analogy to the synthesis of Dex-Mtx in FIG. 20.

In addition to the dihalo linker shown in FIG. 20, we synthesize the Steroid-Mtx CIDs with the linker 1,10-diiododecane, which has also been successfully used to make Dex-Mtx.

Screens

These CID's are screened against a yeast ORF library fused to an activation domain using the yeast three-hybrid screen. This screen can be done using technology already in place at GPC-Biotech. We should start screening immediately with Dex-Mtx to work out any bugs while we are preparing the other Steroid-Mtx compounds.

Results

This screen efficiently picks out both known and unknown steroid-binding proteins.

### BIBLIOGRAPHY

Albrecht, H.; et al. J. Med. Chem. 1990, 33, 77-86.

Amara, J. et al, (1997) PNAS, USA 94, 10618-10623.

Austin D J, et al., Chem Biol. 1994 November, 1(3): 131-6. Review.

Baca, M.; Scanlan, T.; Stephenson, R.; Wells, J. Proc. Natl Acad. USA 1997, 94, 10063-10068.

Banoub, J. (1992) Synthesis of Oligosaccharides of 2-amino-2-deoxy sugars. Chem. Rev. 92, 1167-1195.

Baum E Z, Bebernitz G A, Gluzman Y., “beta-Galactosidase containing a human immunodeficiency virus protease cleavage site is cleaved and inactivated by human immunodeficiency virus protease.” Proc Natl Acad Sci USA. 1990 Dec;87(24): 10023-7. P0 Belshaw P J, et al., Chem Biol., 1996 September, 3(9); 731-8.

Belshaw P J, et al., Proc Natl Acad Sci USA 1996 May 14, 93(10): 4604-7.

Biely, P.; Vrsanska, M.; Tenkanen, M.; Kluepfel, D. (1997) Endo-b-1,4-xylanase families: differences in catalytic properties. J Biotechnol. 57, 151-166.

Blacklock, Thomas J.; Butcher, John W.; Sohar, Paul; Lamanec, Theresa Rothauser; Grabowski, Edward J. J. A versatile synthesis of 1,1-dioxo 7-substituted cephems: preparation of the human leukocyte elastase (HLE) inhibitor 1,1-dioxo-trans-7-methoxycephalosporanic acid tert-butyl ester. J. Org. Chem. 1989, 54(16), 3907-13.

Bolin, J.; Filman, D.; Matthews, D.; Hamlin, R.; Kraut, J. J. Biol. Chem. 1982, 257, 13663-13672.

Boons, G. (1996) Strategies in oligosaccharide synthesis. Tetrahedron 52, 1095-1121.

Braisted, A.; Schultz, P. J. Am. Chem. Soc. 1990, 112, 7430-7431.

Braselmann, S.; Graninger, P.; Busslinger, M. (1993), PNAS, USA 90, 1657-1661.

Cadwell, R.; Joyce, G.; Cold Spring Harbor Laboratory Press; New York, 1994.

Carruthers, W. Cycloaddition Reactions in Organic Synthesis; Pergamon Press: Oxford, 1990; Vol. 8.

Chakraborti, P.; Garabedian, M.; Yamamoto, K.; S S Simons, J. J. Biol. Chem. 1991, 266, 22075-22078.

Chook, Y.; Gray, J.; Ke, H.; Lipscomb, W. J. Mol. Biol. 1994, 240, 476-500.

Choi J. et al., Science, 1996 Jul 12; 273(5272): 239-42.

Clackson, T. et al., (1998) PNAS, USA 95, 10437-10442.

Coleman, R.; Danishefsky, S. 28 1989, 157-161.

Correat, J. U.; Elango, N.; Polacheck, I.; Cabib, E. (1982) Endochitinase, a Mannan-associated Enzyme from Saccharomyces cerevisiae. J. Biol. Chem. 257, 1392-1397.

Crabtree, G.; Schreiber, S. Trends Biochem. Sci. 1996, 21, 418-422.

Crameri, A.; Raillard, S. -A.; Bermudez, E.; Stemmer, W. Nature 1998, 391, 288-91.

Danishefsky, S. J.; McClure, K. F.; Randolph, J. T.; Ruggeri, R. B. (1993) A strategy for the solid-phase synthesis of oligosaccharides. Science 260, 1307-1309.

Dautin N, Karimova G, Ullmann A, Ladant D. “Sensitive genetic screen for protease activity based on a cyclic AMP signaling cascade in Escherichia coli.” J. Bacteriol. 2000 December;182(24):7060-6.

DeGrado, W.; Nilsson, B. Curr. Opin. Struc. Biol. 1997, 7, 455-456, and following articles.

Diver, S T, Journal of the American Chemical Society, 1997 June 4, V119 N22; 5106-5109.

Duchene, A.; et al., Synlett 1994, 524-526.

Durckheimer, W.; Adam, F.; Fischer, G.; Kirrstetter, R. Adv. Drug Res. 1988, 17, 61-234.

Eisenbeis, S.; et al. Proc. Natl. Acad. Sci. USA 1985, 82, 1084-1089.

Fields, S.; Song, O. Nature 1989, 340, 245-246.

Frere, Jean Marie; Joris, Bernard, Penicillin-sensitive enzymes in peptidoglycan biosynthesis, CRC Crit. Rev. Microbio., 1985, 11(4), 299-396.

Fujimoto, Z.; et al. (1998) Crystal structure of a catalytic-site mutant a-amylase from Bacillus subtilis complexed with maltopentaose. J. Mol. Biol. 277, 393-407.

Galleni, M.; Frere, J. Biochem. J. 1988, 255, 119-122.

Galleni, M; Amicosante, G.; Frere, J. Biochem. J. 1988, 255, 123-129.

Galleni, M.; et al. Biochem. J. 1988, 250, 753-760.

Ghuysen, J. (1991) Serine b-lactamases and penicillin-binding proteins. Annu. Rev. Microbiol. 45, 37-67.

Gossen, M.; Bujard, H. (1992) PNAS, USA 89, 5547-5551.

Gossen, M.; Freundlieb, S.; Bender, G.; Muller, G.; Hiller, W.; Bujard, H. (1995), Science 268, 1766-1769.

Gouverneur, V.; et al. Science 1993, 262, 204-208.

Govindan, M.; Manz, B. Eur. J. Biochem. 1980, 108, 47-53.

Gray, J.; Golinelli-Pimpaneau, B.; Knowles, J. Biochemistry 1991, 29, 376-383.

Gray, J.; Eren, D.; Knowles, J. Biochemistry 1990, 29, 8872-8878.

Gyuris, J.; Golemis, E.; Chertkov, H.; Brent, R. Cell 1993, 75, 791-803.

Haynes, M.; Sutra, E.; Hilvert, D.; Wilson, I. Science 1994, 263, 646-652.

Hawkins C J, Wang S L, Hay B A. “A cloning method to identify caspases and their regulators in yeast: identification of Drosophila IAPI as an inhibitor of the Drosophila caspase DCP-I.” Proc. Natl Acad Sci U S A. 1999 March 16;96(6):2885-90.

Hermes, J.; Blacklow, S.; Knowles, J. Proc. Natl. Acad. Sci. USA 1990, 87, 696-700.

Hilvert, D.; Kaiser, E. J. Am. Chem. Soc. 1985, 107, 5805-5806.

Hilvert, D. Curr. Opin. Struct. Biol. 1994, 4, 612-617.

Hilvert, D.; Hill, K.; Nared, K.; Auditor, M. -T. J. Am. Chem. Soc. 1989, 111, 9261-9262.

Ho S N, et al., Nature, 1996 Aug 29; 382(6594): 822-6.

Holsinger L J, et al., Proc Natl Acad Sci USA 1995 October 10, 92(21): 9810-4.

Hu, J.; O'Shea, E.; Kim, P.; Sauer, R. Science 1990, 250, 1400-1403.

Hu, J. Structure 1995, 3, 431-433.

Huang, T.; Barclay, B.; Kalman, T.; vonborstel, R.; Hastings, P. Gene 1992, 121,167-171.

J. Hudson; Fields, S.; et al. (1997) The complete set of predicted genes from Saccharomyces cerevisiae in a readily usable form. Genome Res. 7, 1169-1173.

Hung, D T, Chemistry & Biology, 1996 August, V3 N8: 623-639.

Imperiali, B.; Roy, R. J. Am. Chem. Soc. 1994, 116, 12083-12084.

Jacobsen, J.; et al. Science 1992, 256, 365-367.

Johnson K.; Allemann, R.; Widmer, H.; Benner, S. Nature 1993, 365, 530-532.

Johnsson, N.; Varshavsky, A. Proc. Natl. Acad. Sci. USA 1994, 91, 10340-10344.

Kahne, D.; Walker, S.; Cheng, Y.; Engen, D. V. (1989) Glycosylation of unreactive substrates. J. Am. Chem. Soc 111, 6881-6882.

Kaiser, E.; Lawrence, D. Science 1984, 226, 505-511.

Kamada S, Kusano H, Fujita H, Ohtsu M, Koya R C, Kuzumaki N, Tsujimoto Y. “A cloning method for caspase substrates that uses the yeast two-hybrid system: cloning of the antiapoptotic gene gelsolin.” Proc Natl Acad Sci U S A. 1998 Jul 21;95(15):8532-7.

Karimova, G.; Pidoux, J.; Ullmann, A.; Ladant, D. Proc. Natl. Acad. Sci. USA 1998, 95, 5752-5756.

Kast, P.; Asif-Ullah, M.; Jiang, N.; Hilvert, D. Proc. Natl. Acad. Sci. USA 1996, 93, 5043-5048.

Kelly, J.; et al. (1986) On the origin of bacterial resistance to penicillin: comparison of a b-lactamase and a penicillin target. Science 231, 1429-1437.

Kelly, Judith A.; Kuzin, Alexandre P., The refined crystallographic structure of a DD-peptidase penicillin-target enzyme at 1.6 ANG. resolution. J. Mol. Biol. (1995), 254(2), 223-36.

Knowles, J. Science 1987, 236, 1252-1258.

Koltermann, A.; et al. Proc. Natl. Acad. Sci. USA 1998, 95, 1421-1426.

Klemm, J D, Annu Rev Immunol, 1998, 16:569-92.

Knox, J.; Moews, P.; Frere, J. (1996) Molecular evolution of bacterial b-lactam resistance. Chemistry & Biology 3, 937-947.

Kopytek S J, Standaert R F, Dyer J C, Hu J C., “Chemically induced dimerization of dihydrofolate reductase by a homobifunctional dimer of methotrexate. Chem Biol. 2000 May;7(5):313-21.

Kralovec, J.; Spencer, G.; Blair, A.; Mammen, M.; Singh, M.; Ghose, T. J. Med. Chem. 1989, 32, 2426-2431.

Kuranda, M. J.; Robbins, P. W. (1987) Cloning and heterologous expression of glycosidase genes from Saccharomyces cerevisiae. Proc. Natl. Acad. Sci. USA 84, 2585-2589.

Kuranda, M. J.; Robbins, P. W. (1991) Chitinase is required for cell separation during growth of Saccharomyces cerevisiae. J. Biol. Chem. 266, 19758-19767.

Kuzin, Alexandre P.; Liu, Hansong; Kelly, Judith A.; Knox, James R. Binding of cephalothin and cefotaxime to D-Ala-D-Ala-peptidase reveals a functional basis of a natural mutation in a low-affinity penicillin-binding protein and in extended-spectrum β-lactamases. Biochemistry (1995), 34(29), 9532-40.

Lee, A.; Karplus, P.; Ganem, B.; Clardy, J. J. Am. Chem. Soc. 1995, 117, 3627-3628.

Leung, D.; Chen, E.; Goeddel, D. Technique 1989, 1, 11-15.

Liang, R.; Kahne, D.; el al. (1996) Parallel synthesis and screening of a solid phase carbohydrate library. Science 274, 1520-1522.

Licitra, E.; Liu, J. Proc. Natl. Acad. Sci. USA 1996, 93, 12817-12821.

Lin, Hening; Abida, Wassim M.; Sauer, Robert T.; Cornish, Va. W., Dexamethasone-Methotrexate: An Efficient Chemical Inducer of Protein Dimerization In Vivo. J. Am. Chem. Soc. (2000), 122(17), 4247-4248.

Lobkovsky, E.; et al. Proc. Natl. Acad. Sci. USA 1993, 90, 11257-11261.

Mangia, Alberto; Scandroglio, Angelo; Del Buttero, Paola. Synthesis of tert-butyl ester of 7-aminocephalosporanic acid. Org. Prep. Proced. Int. 1986, 18(1), 13-15.

Manz, B.; Heubner, A.; Kohler, I.; Grill, H. -J.; Pollow, K. Eur. J Biochem. 1983, 131, 333-338.

Martzen, M. R.; McCraith, S. M.; Spinelli, S. L.; Torres, F. M.; Fields, S.; Grayhack, E. J.; Phizicky, E. M. (1999) A biochemichal genomics approach for identifying genes by the activitiy of their products. Science 286, 1153-1155.

Meyer, D.; el al. Bioconjugate Chem. 1995, 6, 440-446.

Monnaie, D.; Virden, R.; Frere, J. FEBS 1992, 306, 108-112.

No, D.; Yao, T.; Evans, R. (1996) PNAS, USA 93, 3346-3351.

Oppolzer, W. In Comp. Org. Syn.; B. Trost and I. Fleming, Eds.; Pergamon Press: New York, 1991; Vol. 5; pp 315-399.

Overman, L; Taylor, G.; Petty, C.; Jessup, P. J. Org. Chem. 1978, 43, 2164-2167.

The chemistry of the β-lactams; Page, M., Ed.; Chapman & Hall: Glasgo, 1992.

Pedersen, H.; et al. Proc. Acad. Sci. USA 1998, 95, 10523-10528.

Picard, D.; Yamamoto, K. EMBO J. 1987, 6, 3333-3338.

Pluckthun, A.; Knowles, J. J. Biol. Chem. 1987, 262, 3951-3957.

Posner, B.; Smiley, J.; Lee, I.; Benkovic, S. Trends Biochem. Sci. 1994, 19, 145-150.

Pruschy, M.; Spencer, D.; Kapoor, T.; Miyake, H.; Crabtree G.; Schreiber, S. Chem. Biol. 1994, 1, 163-172.

Qian, M.; Haser, R.; Buisson, G.; Duee, E.; Payan, F. (1994) The active center of a mammalian alpha-amylase. Structure of the complex of a pancreatic alpha-amylase with a carbohydrate inhibitor refined to 2.2-Å resolution. Biochemistry 33, 6284-94.

Reidhaar-Olson, J.; et al. Methods Enz. 1991, 208, 564-586.

Robbins, P.; Trimble, R.; Wirth, D.; Hering, C.; Maley, F.; Maley, G.; Das, R.; Gibson, B.; Royal, N.; Biemann, K. (1984) Primary structure of the Streptomyces enzyme endo-beta-N-acetylglucosaminidase H. J. Biol. Chem. 259, 7577-7583.

Rossi, R.; Charlton, C.; Blau, H. Proc. Natl. Acad. Sci. 1997, 94, 8405-8410.

Rosen, M.; Schreiber, S. (1992),Angew. Chem. Int. Ed. Engl. 31, 384-400.

Sasso, S.; Gilli, R.; Sari, J.; Rimet, O.; Briand, C. Biochim. Biophys. Acta 1994, 1207, 74-79.

Schmidt, R., Angew. Chem. Int. Ed. Engl.(1986) 25, 212-235.

Schreiber, S L, Bioorg Med Chem 1998 August;6(8): 1127-52.

Schultz, P. Ang. Chem. Int. Ed. Eng. 1989, 28, 1283-1444.

Schultz, P.; Lerner, R. Science 1995, 269, 1835-1842.

Seeberger, P. H.; Danishefsky, S. J. (1998) Solid-phase synthesis of oligosaccharides and glycoconjugates by the glycal assembly method: A five year retrospective. Acc. Chem. Res 31, 685-695.

Shokat, K.; Leumann, C.; Sugasawara, R.; Schultz, P. Science 1989, 338, 269-272.

Smith T A, Kohorn B D. “Direct selection for sequences encoding proteases of known specificity.” Proc Nati Acad Sci U S A. 1991 Jun 15;88(12):5159-62.

Sogaard, M.; Kadziola, A.; Haser, R.; Svensson, B. (1993) Site-directed mutagenesis of His93, Aspl 80, Glu205, His290, Asp291 at the active site and Trp279 at the raw starch binding site in Barley a-amylase I. J. Biol. Chem. 268, 22480-22484.

Spencer D M, et al., Curr Biol. 1996 Jul 1, 6(7): 839-47.

Spencer D M, et al., Proc Natl Acad Sci USA 1995 Oct 10, 92(21): 9805-9.

Spencer, D.; Wandless, T.; Schreiber, S.; Crabtree, G. Science 1993, 262, 1019-1024.

Stemmer, W. Proc. Natl. Acad. Sci. USA 1994, 91, 10747-10751.

Stemmer, W. Nature 1994, 370, 389-391.

Stockwell, B R, Chem Biol., 1998 Jul 5(7): 385-95.

Stockwell, B R, Curr Biol., 1998 Jun 18; 8(13): 761-70.

Suckling, C.; et al. Biorg. Med. Chem. Lett. 1992, 2, 49-53.

Tarentino, A.; Quinones, G.; Trumble, A.; Changchien, L.; Duceman, B.; Maley, F. (1990) Molecular cloning and amino acid sequence of peptide-N4-(N-acetyl-beta- D-glucosaminyl)asparagine amidase from Flavobacterium meningosepticum. J. Biol. Chem. 265, 6961-6966.

Tarentino, A.; T H Plummer, J.; et al. (1992) Multiple endoglycosidase (Endo) F activities expressed by Flavobacterium meningosepticum. J. Biol. Chem. 267, 3868-3872.

Toshima, K.; Tatsuta, K. (1993) Recent progress in O-glycosylation methods and its application to natural products synthesis. Chem. Rev. 93, 1503-1531.

Trimble, R.; Tarentino, A. (1991) Identification of distinct endoglycosidase (Endo) activities in Flavobacterium meningosepticum: Endo F1, Endo F2, and Endo F3. J. Biol. Chem. 266, 1646-1651.

Varki, A. (1993) Biological roles of oligosaccharides: all the theories are correct. Glycobiology 3, 97-130.

Vihinen, M.; Mantsala, P.; et al. (1990) Site-directed mutagenesis of a thermostable a-amylase from Bacillus stearothermophilus: putative role of three conserved residues. J. Biochem. 107, 267-272.

Vrudhula, V.; Svensson, H.; Senter, P. J. Med. Chem. 1995, 38, 1380-1385.

Wagner, J.; Lerner, R.; Barbas, C. Science 1995, 270, 1797-1800.

Wagner, R.; Rhoades, T.; Or, Y.; Lane, B.; Hsieh, G.; Mollison, K.; Luly, J. J. Med. Chem. 1998, 41, 1764-1776.

Wang, Y.; BW O'Malley, J.; Tsai, S.; O'Malley, B. (1994) PNAS, USA 91, 8180-8184.

Wells, J.; Vasser, M.; Powers, D. Gene 1985, 34, 315-323.

Wharton, R.; Patashne, M. Nature 1985, 316, 601-605.

Wiegand, G.; Epp, O.; Huber, R. (1995) The crystal structure of porcine pancreatic a-amylase in complex with the microbial inhibitor tendamistat. J. Mol. Biol. 247, 99-110.

Wilcox, E.; Whitaker, J. (1984) Some aspects of the mechanism of complexation of red kidney bean a-amylase inhibitor and a-maylase. Biochemistry 23, 1783-1791.

Wilks, H.; et al. Science 1988, 242, 1541-1544.

Wong, K.; Tan, L.; Saddler, J. (1988) Multiplicity of b-1,4-zylanase in microorganisms: Functions and applications. Microbiol. Rev. 52, 305-317.

Yan, L.; Taylor, C. M.; R. Goodnow, J.; Kahne, D. (1994) Glycosylation on the Merrifield resin using anomeric sulfoxides. J. Amer. Chem. Soc. 116, 6953-6954.

Yang J., Curr Biol 1998, January 1, 8(1): 11-8.

Ye, X.; Wong, C. (2000) Anomeric reactivity-based one-pot oligosaccharide synthesis: A rapid route to oligosaccharide libraries. J. Org. Chem. 65, 2410-2431.

Zhang, J.; Dawes, G.; Stemmer, W. Proc. Natl. Acad. Sci. USA 1997, 94,4504-4509.

Zhang, Z.; Ollmann, I. R. (1999) Programmable one-pot oligosaccharide synthesis. J. Amer. Chem. Soc. 121, 134-153.

Zlokarnik, G.; et al. (1998) Quantitation of transcription and clonal selection of single living cells with a b-lactamase reporter. Science 279, 84-88

Zoller, M.; Smith, M. Methods Enz. 1983, 200, 468-500.

What is claimed is:

1. A compound having the formula:
H1-Y-H2
wherein H1 is a substrate capable of selectively binding to a first receptor;
wherein H2 is a substrate capable of selectively binding to and selectively forming a covalent bond with a second receptor; and
wherein Y is a moiety providing a covalent linkage between H1 and H2, which may be present or absent, and when absent, H1 is covalently linked to H2.

2. The compound of claim 1, wherein H1 is a Methotrexate moiety or an analog thereof.

3. The compound of claim 1, wherein H2 is a cephem moiety capable of selectively binding to and selectively forming a covalent bond with the penicillin-binding-protein (“PBP”).

4. The compound of claim 1, wherein H2 is a fluorouracil moiety capable of selectively binding to and selectively forming a covalent bond with the thymidine synthase (“TS”) enzyme.

5. The compound of claim 2 having the structure:

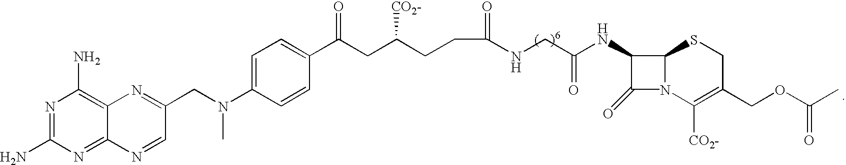

6. A complex between the compound of claim 1 and a fusion protein, the fusion protein comprising a receptor domain which binds to the compound.

7. The complex of claim 6 wherein the fusion protein further comprises a DNA-binding domain fused to the receptor domain.

8. The complex of claim 6 wherein the fusion protein further comprises a transcription activation domain fused to the receptor domain.

9. The complex of claim 6, wherein the receptor domain is dihydrofolate reductase (“DHFR”), penicillin-binding-protein (“PBP”), or thymidine synthase (“TS”) enzyme.

10. The complex of claim 9, wherein the PBP is the Streptomyces R61 PBP.

11. The complex of claim 9, wherein the DHFR is the E.coli DHFR (“eDHFR”).

12. The complex of claim 7, wherein the fusion protein is eDHFR-LexA or R61-LexA.

13. The complex of claim 8, wherein the fusion protein is eDHFR-B42 or R61-B42.

14. A cell comprising the complex of claim 6.

15. A cell comprising a DNA sequence which on transcription
gives rise to a first fusion protein exogenous to the cell and a second fusion protein exogenous to the cell,
wherein the first fusion protein is a receptor domain fused with a DNA-binding domain; and
wherein the second fusion protein is a transcription activation domain fused to either a penicillin-binding-protein (“PBP”) or to a thymidine synthase (“TS”) enzyme.

16. The cell of claim 15, wherein the receptor domain of the first fusion protein is DHFR.

17. The cell of claim 15, wherein the DNA-binding domain of the first fusion protein is LexA.

18. The cell of claim 15, wherein the transcription activation domain of the second fusion protein is B42.

19. The cell of claim 15, wherein the PBP is the Streptomyces R61 PBP.

20. The cell of claim 15, wherein the first fusion protein is eDHFR-LexA, and the second fusion protein is R61-B42.

21. The cell of claim 15, where the cell is a yeast cell, a bacteria cell or a mammalian cell.

22. The cell of claim 15, where the cell is S. cerevisiae or E. coli.

23. A method of dimerizing two fusion proteins inside a cell
using the compound of claim 1, comprising the steps of
a) providing a cell that expresses a first fusion protein which comprises a receptor domain that binds to Hi, and a second fusion protein which comprises a receptor domain that binds to and forms a covalent bond with H2, and
b) contacting the compound with the cell so as to dimerize the two fusion proteins.

24. The method of claim 23, wherein the receptor domain of the first fusion protein is DHFR.

25. The method of claim 23, wherein the DNA-binding domain of the first fusion protein is LexA.

26. The method of claim 23, wherein the transcription activation domain of the second fusion protein is B42.

27. The method of claim 23, wherein the receptor domain of the second fusion protein is a penicillin-binding-protein (“PBP”) or to a thymidine synthase (“TS”) enzyme.

28. The method of claim 27, wherein the PBP is the Streptomyces R61 PBP.

29. The method of claim 23, wherein the first fusion protein is eDHFR-LexA, and the second fusion protein is R61-B42.

30. A method for identifying a molecule that binds a known target in a cell from a pool of candidate molecules, comprising:
(a) forming a screening molecule by covalently bonding each molecule in the pool of candidate molecules to a substrate capable of selectively binding to and selectively forming a covalent bond with a receptor;
(b) introducing the screening molecule into a cell culture comprising cells that express
a first fusion protein of a DNA-binding domain fused to a known target receptor domain against which the candidate molecule is screened,
a second fusion protein which comprises a receptor domain capable of binding to and forming a covalent bond with the screening molecule, and
a reporter gene wherein expression of the reporter gene is conditioned on the proximity of the first fusion protein to the second fusion protein;
(c) permitting the the screening molecule to bind to the first fusion protein and to the second fusion protein, bringing the two fusion proteins in to proximity so as to activate the expression of the reporter gene;
(d) selecting the cell that expresses the reporter gene; and
(e) identifying the small molecule that binds the known target receptor.

31. The method of claim 30, wherein the cell is selected from the group consisting of insect cells, yeast cells, mammalian cells, and their lysates.

32. The method of claim 30, wherein the DNA-binding domain of the first fusion protein is LexA.

33. The method of claim 30, wherein the transcription activation domain of the second fusion protein is B42.

34. The method of claim 30, wherein the receptor domain of the second fusion protein is a penicillin-binding-protein (“PBP”) or to a thymidine synthase (“TS”) enzyme.

35. The method of claim 34, wherein the PBP is the Streptomyces R61 PBP.

36. The method of claim 30, wherein the molecule is obtained from a combinatorial library.

37. The method of claim 30, wherein the steps (b)-(e) of the method are iteratively repeated in the presence of a preparation of random small molecules for competitive binding with the screening molecule so as to identify a molecule capable of competitively binding the known target receptor.

38. A method for identifying an unknown target receptor to
which a molecule is capable of binding in a cell, comprising:
(a) providing a screening molecule having a ligand which has a specificity for the unknown target receptor covalently bonded to a substrate capable of selectively binding to and selectively forming a covalent bond with a receptor;
(b) introducing the screening molecule into a cell which expresses
a first fusion protein of a DNA-binding domain fused to the unknown target receptor domain against which the candidate molecule is screened,
a second fusion protein which comprises a receptor domain capable of binding to and forming a covalent bond with the screening molecule, and
a reporter gene wherein expression of the reporter gene is conditioned on the proximity of the first fusion protein to the second fusion protein;
(c) permitting the screening molecule to bind to the first fusion protein and to the second fusion protein so as to activate the expression of the reporter gene;
(d) selecting which cell expresses the unknown target receptor; and
(e) identifying the unknown target receptor.

39. The method of claim 38, wherein the unknown protein target is encoded by a DNA from the group consisting of genomicDNA, cDNA and syntheticDNA.

40. The method of claim 38, wherein the ligand has a known biological function.

41. A compound having the formula:
H1-Y-H2
wherein H1 is Mtx or an analog thereof;
wherein H2 is a substrate capable of binding to a receptor, and
wherein Y is a moiety providing a covalent linkage between H1 and H2, which may be present or absent, and when absent, H1 is covalently linked to H2.

42. The compound of claim 41, having the formula:

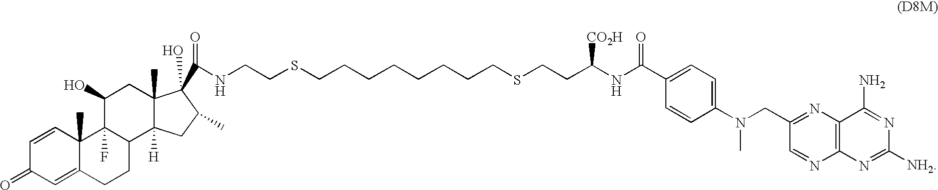

43. The compound of claim 41, having the formula:

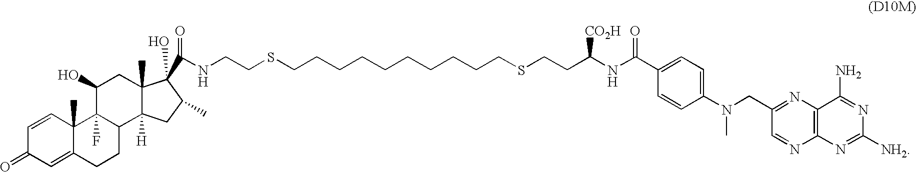

44. The compound of claim 41, having the formula:

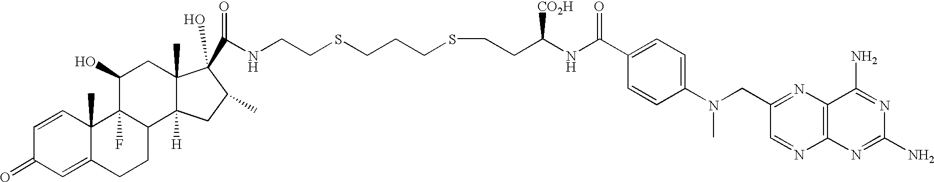

45. The compound of claim 41, having the formula:

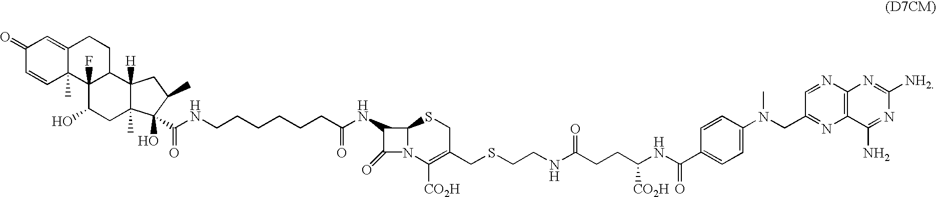

46. The compound of claim 41, having the formula:

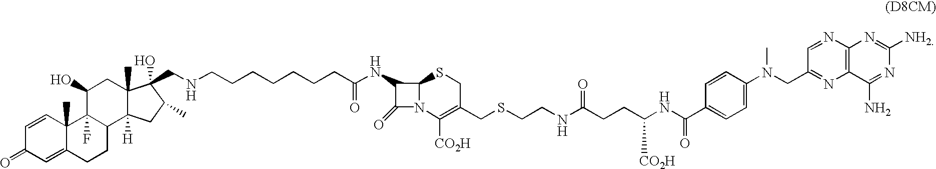

47. A complex between the compound of claim 41 and a fusion protein which comprises a binding domain capable of binding to methotrexate, wherein H1 of the compound binds to the binding domain of the fusion protein.

48. The complex of claim 47, wherein the binding domain is that of the DHFR receptor.

49. The complex of claim 47, wherein the fusion protein is DHFR-LexA.

50. The complex of claim 47, wherein the fusion protein is DHFR-B42.

51. A cell comprising the complex of claim 47.

52. A method for screening a cDNA library by identifying the expressed protein target, comprising:
(a) providing a screening molecule comprising a methotrexate moiety or an analog of methotrexate covalently bonded to a ligand which has a known specificity;
(b) introducing the screening molecule into a cell which expresses a first fusion protein comprising a binding domain capable of binding methotrexate, a second fusion protein comprising the expressed unknown protein target, and a reporter gene wherein expression of the reporter gene is conditioned on the proximity of the first fusion protein to the second fusion protein;
(c) permitting the screening molecule to bind to the first fusion protein and to the second fusion protein so as to activate the expression of the reporter gene;
(d) selecting which cell expresses the reporter gene; and
(e) identifying the unknown protein target and the corresponding cDNA.

53. The method of claim 52, wherein the unknown protein target is encoded by a DNA from the group consisting of genomicDNA, cDNA and syntheticDNA.

54. A new protein cloned by the method of claim 52.
